(19) United States
(12) Patent Application Publication
(10) Pub. No.: US 2009/0012066 A1
(43) Pub. Date: Jan. 8, 2009

(54) Method of Use of Deacetylase Inhibitors

(75) Inventors: Seigo Izumo, Brookline, MA (US); Suraj Shivappa Shetty, Andover, MA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG

(21) Appl. No.: 12/063,141

(22) PCT Filed: Aug. 9, 2006
(86) PCT No.: PCT/US2006/030877

§ 371 Date: Jul. 9, 2008

Related U.S. Application Data
(60) Provisional application No. 60/707,137, filed on Aug. 10, 2005.

(51) Int. Cl. A61K 31/551; A61K 31/403; A61K 31/4166; A61K 31/5375; A61K 31/437; A61K 31/495

(52) U.S. Cl. 514/221; 514/415; 514/389; 514/300; 514/255.03; 514/239.5

(57) ABSTRACT

The present invention provides methods of treating and/or preventing pathologic cardiac hypertrophy and heart failure comprising administering hydroxamate compounds which are deacetylase inhibitors.

The present invention relates to hydroxamate compounds which are inhibitors of histone deacetylase. The inventive compounds are useful as pharmaceuticals for the treatment and/or prevention of cardiac hypertrophy and heart failure.

### BACKGROUND

Reversible acetylation of histones is a major regulator of gene expression that acts by altering accessibility of transcription factors to DNA. In normal cells, histone deacetylase (HDA) and histone acetyltransferase together control the level of acetylation of histones to maintain a balance. Inhibition of HDA results in the accumulation of hyperacetylated histones, which results in a variety of cellular responses.

Inhibitors of HDA have been studied for their therapeutic effects on cancer cells. For example, butyric acid and its derivatives, including sodium phenylbutyrate, have been reported to induce apoptosis in vitro in human colon carcinoma, leukemia and retinoblastoma cell lines. However, butyric acid and its derivatives are not useful pharmacological agents because they tend to be metabolized rapidly and have a very short half-life in vivo. Other inhibitors of HDA that have been widely studied for their anti-cancer activities are trichostatin A and trapoxin. Trichostatin A is an antifungal and antibiotic and is a reversible inhibitor of mammalian HDA. Trapoxin is a cyclic tetrapeptide, which is an irreversible inhibitor of mammalian HDA. Although trichostatin and trapoxin have been studied for their anti-cancer activities, the in vivo instability of the compounds makes them less suitable as anti-cancer drugs.

Inhibitors of HDA have also been studied for their therapeutic effects on pathological cardiac hypertrophy and heart failure. Transgenic mice that over-express Hop, a homeodomain protein expressed by cardiac myocytes, develop severe cardiac hypertrophy, cardiac fibrosis, and premature death. Treatment of these animals with trichostatin A, an HDA inhibitor, prevents cardiac hypertrophy (Kook et al. 2003). In addition, trichostatin A also attenuates hypertrophy induced by infusion of isoproterenol. The in vivo instability of trichostatin makes it less suitable as a treatment option for heart failure. Thus, there exists a strong need for active agents that are suitable for treating and/or preventing pathological cardiac hypertrophy and ameliorating or reversing the biochemical processes that lead to heart failure and death.

### SUMMARY

The present invention provides efficacious deacetylase inhibitor compounds that are useful as pharmaceutical agents having the formula (I):

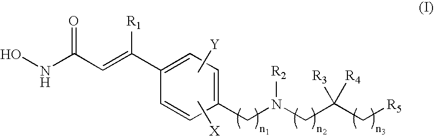

wherein
R₁ is H, halo, or a straight chain C₁-C₆ alkyl (especially methyl, ethyl or n-propyl, which methyl, ethyl and n-propyl substituents are unsubstituted or substituted by one or more substituents described below for alkyl substituents);
R₂ is selected from H, C₁-C₁₀ alkyl, (e.g. methyl, ethyl or —CH₂CH₂—OH), C₄-C₉ cycloalkyl, C₄-C₉ heterocycloalkyl, C₄-C₉ heterocycloalkylalkyl, cycloalkylalkyl (e.g., cyclopropylmethyl), aryl, heteroaryl, arylalkyl (e.g. benzyl), heteroarylalkyl (e.g. pyridylmethyl), —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl (e.g. benzyl), heteroarylalkyl (e.g. pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, such as CH₃ and CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl (e.g., cyclopropylmethyl), aryl, heteroaryl, arylalkyl (e.g., benzyl, 2-phenylethenyl), heteroarylalkyl (e.g., pyridylmethyl), OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl (e.g., benzyl), and heteroarylalkyl (e.g., pyridylmethyl);
R₉ is selected from C₁-C₄ alkyl, for example, CH₃ and CF₃, C(O)-alkyl, for example C(O)CH₃, and C(O)CF₃;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl (e.g., benzyl), heteroarylalkyl (e.g., pyridylmethyl), amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycles, heteroaryl, arylalkyl, heteroarylalkyl, polyheteroaryl and NR₁₃R₁₄;
m is an integer selected from 0 to 6; and
Z is selected from O, NR₁₃, S and S(O),
   or a pharmaceutically acceptable salt thereof.

The compounds of the present invention are suitable as active agents in pharmaceutical compositions that are efficacious particularly for treating and/or preventing pathological cardiac hypertrophy and heart failure. The pharmaceutical composition has a pharmaceutically effective amount of the present active agent along with other pharmaceutically acceptable excipients, carriers, fillers, diluents and the like. The term pharmaceutically effective amount as used herein indicates an amount necessary to administer to a host to achieve a therapeutic result, especially an an inhibitory effect on pathological cardiac hypertrophy and heart failure, e.g., inhibition of pathologically hypertrophied cardiac cells and its adverse consequences including heart failure and arrhythmogenesis.

### DETAILED DESCRIPTION

The present invention provides hydroxamate compounds, e.g., hydroxamic acids, that are inhibitors of deacetylases, preferably inhibitors of histone deacetylases. The hydroxamate compounds are highly suitable for treating and/or preventing pathological cardiac hypertrophy and heart failure. The hydroxamate compounds of the present invention have the following structure (I):

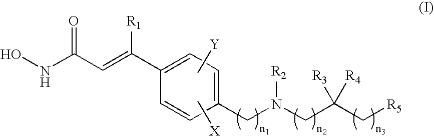

wherein
R₁ is H, halo, or a straight chain C₁-C₆ alkyl (especially methyl, ethyl or n-propyl, which methyl, ethyl and n-propyl substituents are unsubstituted or substituted by one or more substituents described below for alkyl substituents);
R₂ is selected from H, C₁-C₁₀ alkyl, (preferably C₁-C₆ alkyl, e.g. methyl, ethyl or —CH₂CH₂—OH), C₄-C₉ cycloalkyl, C₄-C₉ heterocycloalkyl, C₄-C₉ heterocycloalkylalkyl, cycloalkylalkyl (e.g., cyclopropylmethyl), aryl, heteroaryl, arylalkyl (e.g. benzyl), heteroarylalkyl (e.g. pyridylmethyl), —(CH₂)ₙC(O)R₆, —(CH₂)ₙOC(O)R₆, amino acyl, HON—C(O)—CH═C(R₁)-aryl- alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl (e.g. benzyl), heteroarylalkyl (e.g. pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, such as CH₃ and CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl (e.g., cyclopropylmethyl), aryl, heteroaryl, arylalkyl (e.g., benzyl, 2-phenylethenyl), heteroarylalkyl (e.g., pyridylmethyl), OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl (e.g., benzyl), and heteroarylalkyl (e.g., pyridylmethyl);
R₉ is selected from C₁-C₄ alkyl, for example, CH₃ and CF₃, C(O)-alkyl, for example C(O)CH₃, and C(O)CF₃;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ re the same or different and independently selected from H, C₁-C₆ alkyl, C₄-C₉ cycloalkyl, C₄-C₉ heterocycloalkyl, aryl, heteroaryl, arylalkyl (e.g., benzyl), heteroarylalkyl (e.g., pyridylmethyl), amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₈ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycles, heteroaryl, arylalkyl, heteroarylalkyl, polyheteroaryl and NR₁₃R₁₄;
m is an integer selected from 0 to 6; and
Z is selected from O, NR₁₃, S and S(O),
   or a pharmaceutically acceptable salt thereof.

As appropriate, unsubstituted means that there is no substituent or that the only substituents are hydrogen.

Halo substituents are selected from fluoro, chloro, bromo and iodo, preferably fluoro or chloro.

Alkyl substituents include straight and branched C₁-C₆alkyl, unless otherwise noted. Examples of suitable straight and branched C₁-C₆alkyl substituents include methyl, ethyl, n-propyl, 2-propyl, n-butyl, sec-butyl, t-butyl, and the like. Unless otherwise noted, the alkyl substituents include both unsubstituted alkyl groups and alkyl groups that are substituted by one or more suitable substituents, including unsaturation (i.e. there are one or more double or triple C—C bonds), acyl, cycloalkyl, halo, oxyalkyl, alkylamino, aminoalkyl, acylamino and OR₁₅, for example, alkoxy. Preferred substituents for alkyl groups include halo, hydroxy, alkoxy, oxyalkyl, alkylamino, and aminoalkyl.

Cycloalkyl substituents include C₃-C₉ cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆ alkyl, halo, hydroxy, aminoalkyl, oxyalkyl, alkylamino, and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

Heterocycloalkyl substituents include 3 to 9 membered aliphatic rings, such as 4 to 7 membered aliphatic rings, containing from one to three heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane. Unless otherwise noted, the rings are unsubstituted or substituted on the carbon atoms by one or more suitable substituents, including C₁-C₆ alkyl, C₄-C₉ cycloalkyl, aryl, heteroaryl, arylalkyl (e.g., benzyl), and heteroarylalkyl (e.g., pyridylmethyl), halo, amino, alkyl amino and OR₁₅, for example alkoxy. Unless otherwise noted, nitrogen heteroatoms are unsubstituted or substituted by H, C₁-C₄ alkyl, arylalkyl (e.g., benzyl), and heteroarylalkyl (e.g., pyridylmethyl), acyl, aminoacyl, alkylsulfonyl, and arylsulfonyl.

Cycloalkylalkyl substituents include compounds of the formula —(CH₂)ₙ₅-cycloalkyl wherein n5 is a number from 1-6. Suitable alkylcycloalkyl substituents include cyclopentylmethyl-, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents, including C₁-C₆ alkyl, cycloalkylalkyl (e.g., cyclopropylmethyl), O(CO)alkyl, oxyalkyl, halo, nitro, amino, alkylamino, aminoalkyl, alkyl ketones, nitrile, carboxyalkyl, alkylsulfonyl, aminosulfonyl, arylsulfonyl, and OR₁₅, such as alkoxy. Preferred substituents include including C₁-C₆ alkyl, cycloalkyl (e.g., cyclopropylmethyl), alkoxy, oxyalkyl, halo, nitro, amino, alkylamino, aminoalkyl, alkyl ketones, nitrile, carboxyalkyl, alkylsulfonyl, arylsulfonyl, and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents, including C₁-C₆ alkyl, alkylcycloalkyl (e.g., cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and OR₁₅, such as alkoxy.

Heteroaryl substituents include compounds with a 5 to 7 member aromatic ring containing one or more heteroatoms, for example from 1 to 4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, for example by R₁₃; especially useful N substituents include H, C₁-C₄ alkyl, acyl, aminoacyl, and sulfonyl.

Arylalkyl substituents include groups of the formula —(CH₂)ₙ₅-aryl, —(CH₂)ₙ₅₋₁—(CHaryl)-(CH₂)ₙ₅-aryl or —(CH₂)ₙ₅₋₁CH(aryl)(aryl) wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl wherein heteroaryl and n5 are defined above and the bridging group is linked to a carbon or a nitrogen of the heteroaryl portion, such as 2-, 3- or 4-pyridylmethyl, imidazolylmethyl, quinolylethyl, and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅ wherein n, R₁₃, R₁₄ and R₅ are described above. Suitable aminoacyl substituents include natural and non-natural amino acids such as glycinyl, D-tryptophanyl, L-lysinyl, D- or L-homoserinyl, 4-aminobutryic acyl, ±-3-amin-4-hexenoyl.

Non-aromatic polycycle substituents include bicyclic and tricyclic fused ring systems where each ring can be 4-9 membered and each ring can contain zero, 1 or more double and/or triple bonds. Suitable examples of non-aromatic polycycles include decalin, octahydroindene, perhydrobenzocycloheptene, perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4-9 membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene, 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatom, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, for example by R₁₃; especially useful N substituents include H, C₁-C₄ alkyl, acyl, aminoacyl, and sulfonyl.

Non-aromatic polyheterocyclic substituents include bicyclic and tricyclic fused ring systems where each ring can be 4-9 membered, contain one or more heteroatom, for example, 1, 2, 3, or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Mixed aryl and non-aryl polyheterocycles substituents include bicyclic and tricyclic fused ring systems where each ring can be 4-9 membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents, including, —N—OH, ═N—OH, alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, for example methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W, and —N(R₁₂)C(O)—NHOH and R₁₂ and W are defined above.

The R₂ substituent HON—C(O)—CH═C(R₁)-aryl-alkyl- is a group of the formula

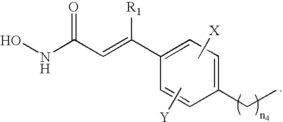

Preferences for each of the substituents include the following:
R₁ is H, halo, or a straight chain C₁-C₄ alkyl;
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl, and —(CH₂)ₙR₇;
R₃ and R₄ are the same or different and independently selected from H, and C₁-C₆ alkyl, or R₃ and R₄ together with the carbon to which they are bound represent C═O, C═S, or C═NR₈;
R₅ is selected from H, C₁-C₆ alkyl, C₄-C₉ cycloalkyl, C₄-C₉ heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄ alkyl, CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, aryl, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl and amino acyl;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, heteroaryl, arylalkyl, heteroarylalkyl and NR₁₃R₁₄;
m is an integer selected from 0 to 6; and
Z is selected from O, NR₁₃, S, S(O),
   or a pharmaceutically acceptable salt thereof.

Useful compounds of the formula (I) include those wherein each of R₁, X, Y, R₃, and R₄ is H, including those wherein one of n₂ and n3 is zero and the other is 1, especially those wherein R₂ is H or —CH₂—CH₂—OH.

One suitable genus of hydroxamate compounds are those of formula Ia:

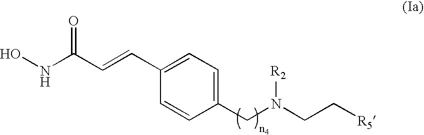

wherein
n₄ is 0-3,
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl and —(CH₂)ₙR₇;
R′₅ is heteroaryl, heteroarylalkyl (e.g., pyridylmethyl), aromatic polycycles, non-aromatic polycycles, mixed aryl and non-aryl polycycles, polyheteroaryl, or mixed aryl and non-aryl polyheterocycles,
   or a pharmaceutically acceptable salt thereof.

Another suitable genus of hydroxamate compounds are those of formula Ia:

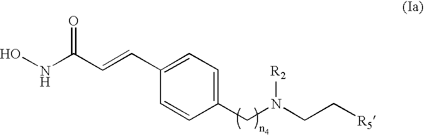

wherein
n₄ is 0-3,
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl and —(CH₂)ₙR₇;
R′₅ is aryl, arylalkyl, aromatic polycycles, non-aromatic polycycles, and mixed aryl and non-aryl polycycles; especially aryl, such as p-fluorophenyl, p-chlorophenyl, p-O—C₁-C₄-alkylphenyl, such as p-methoxyphenyl, and p-C₁-C₄-alkylphenyl; and arylalkyl, such as benzyl, ortho, meta or para-fluorobenzyl, ortho, meta or para-chlorobenzyl, ortho, meta or para-mono, di or tri-O—C₁-C₄-alkylbenzyl, such as ortho, meta or para-methoxybenzyl, m,p-diethoxybenzyl, o,m,p-triimethoxybenzyl, and ortho, meta or para-mono, di or tri C₁-C₄-alkylphenyl, such as p-methyl, m,m-diethylphenyl,
   or a pharmaceutically acceptable salt thereof.

Another interesting genus are the compounds of formula Ib:

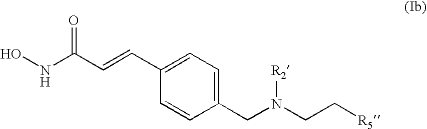

wherein

R′₂ is selected from H, C₁-C₆ alkyl, C₄-C₆ cycloalkyl, cycloalkylalkyl (e.g., cyclopropylmethyl), (CH₂)₂₋₄OR₂₁ where R₂₁ is H, methyl, ethyl, propyl, and i-propyl, and

R″₅ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl,

or a pharmaceutically acceptable salt thereof.

Another interesting genus of hydroxamate compounds are the compounds of formula (Ic)

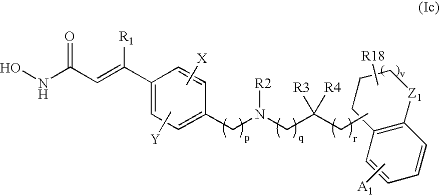

wherein
the ring containing Z₁ is aromatic or non-aromatic, which non-aromatic rings are saturated or unsaturated,
Z₁ is O, S or N—R₂₀,
R18 is H, halo, C₁-C₆alkyl (methyl, ethyl, t-butyl), C₃-C₇cycloalkyl, aryl, for example unsubstituted phenyl or phenyl substituted by 4-OCH₃ or 4-CF₃, or heteroaryl, such as 2-furanyl, 2-thiophenyl or 2-, 3- or 4-pyridyl;
R₂₀ is H, C₁-C₆alkyl, C₁-C₆alkyl-C₃-C₉cycloalkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl)
A₁ is 1, 2 or 3 substituents which are independently H, C₁-C₆alkyl, —OR₁₉, halo, alkylamino, aminoalkyl, halo, or heteroarylalkyl (e.g., pyridylmethyl),
R₁₉ is selected from H, C₁-C₆alkyl, C₄-C₉cycloalkyl, C₄-C₉heterocycloalkyl, aryl, heteroaryl, arylalkyl (e.g., benzyl), heteroarylalkyl (e.g., pyridylmethyl) and —(CH₂CH═CH(CH₃)(CH₂))₁₋₃H;
R₂ is selected from H, C₁-C₆ alkyl, C₄-C₉ cycloalkyl, C₄-C₉ heterocycloalkyl, alkylcycloalkyl, aryl, heteroaryl, arylalkyl, heteroarylalkyl, —(CH₂)ₙC(O)R₆, amino acyl and —(CH₂)ₙR₇;
v is 0, 1 or 2,
p is 0-3, and
q is 1-5 and r is 0 or
q is 0 and r is 1-5,
   or a pharmaceutically acceptable salt thereof. The other variable substituents are as defined above.

Especially useful compounds of formula (Ic) are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id)

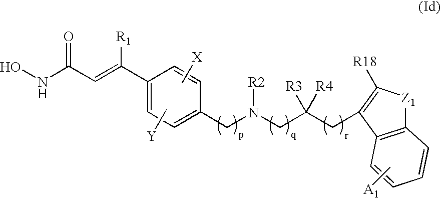

wherein

### Z₁ is O, S or N—R₂₀,

R18 is H, halo, C₁-C₆alkyl (methyl, ethyl, t-butyl), C₃-C₇cycloalkyl, aryl, for example, unsubstituted phenyl or phenyl substituted by 4-OCH₃ or 4-CF₃, or heteroaryl,
R₂₀ is H, C₁-C₆alkyl, C₁-C₆alkyl-C₃-C₉cycloalkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl),
A₁ is 1, 2 or 3 substituents which are independently H, C₁-C₆alkyl, —OR₁₉, or halo,
R₁₉ is selected from H, C₁-C₆alkyl, C₄-C₉cycloalkyl, C₄-C₉heterocycloalkyl, aryl, heteroaryl, arylalkyl (e.g., benzyl), and heteroarylalkyl (e.g., pyridylmethyl);
p is 0-3, and
q is 1-5 and r is 0 or
q is 0 and r is 1-5,
or a pharmaceutically acceptable salt thereof. The other variable substituents are as defined above.

Especially useful compounds of formula (Id) are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (Ie)

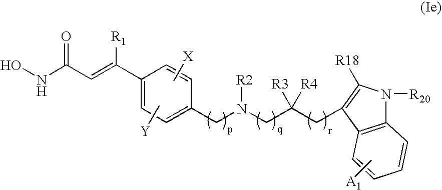

or a pharmaceutically acceptable salt thereof. The variable substituents are as defined above.

Especially useful compounds of formula (Ie) are those wherein R18 is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl (e.g., pyridyl) ring.

Another group of useful compounds of formula (Ie) are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Another group of useful compounds of formula (Ie) are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H, or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula Ie wherein R₂₀ is H or C₁-C₆alkyl, especially H, are important members of each of the subgenuses of compounds of formula Ie described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof, are important compounds of formula (Ie).

The present invention further relates to the compounds of the formula (If):

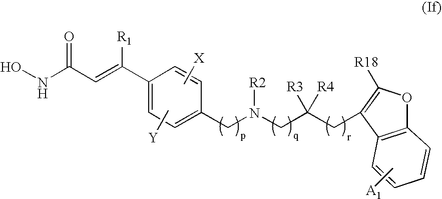

or a pharmaceutically acceptable salt thereof. The variable substituents are as defined above.

Useful compounds of formula (If) are include those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

As is evident to those skilled in the art, the many of the deacetylase inhibitor compounds of the present invention contain asymmetric carbon atoms. It should be understood, therefore, that the individual stereoisomers are contemplated as being included within the scope of this invention.

The hydroxamate compounds of the present invention can be produced by known organic synthesis methods. For example, the hydroxamate compounds can be produced by reacting methyl 4-formyl cinnamate with tryptamine and then converting the reactant to the hydroxamate compounds. As an example, methyl 4-formyl cinnamate 2, is prepared by acid catalyzed esterification of 4-formylcinnamic acid 3 (Bull. Chem. Soc. Jpn. 1995; 68:2355-2362). An alternate preparation of methyl 4-formyl cinnamate 2 is by a Pd-catalyzed coupling of methyl acrylate 4 with 4-bromobenzaldehyde 5.

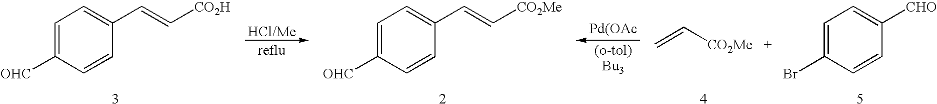

Additional starting materials can be prepared from 4-carboxybenzaldehyde 6, and an exemplary method is illustrated for the preparation of aldehyde 9, shown below. The carboxylic acid in 4-carboxybenzaldehyde 6 can be protected as a silyl ester (e.g., the t-butyldimethylsilyl ester) by treatment with a silyl chloride (e.g., t-butyldimethylsilyl chloride) and a base (e.g. triethylamine) in an appropriate solvent (e.g., dichloromethane). The resulting silyl ester 7 can undergo an olefination reaction (e.g., a Horner-Emmons olefination) with a phosphonate ester (e.g., triethyl 2-phosphonopropionate) in the presence of a base (e.g., sodium hydride) in an appropriate solvent (e.g., tetrahydrofuran (THF)). Treatment of the resulting diester with acid (e.g., aqueous hydrochloric acid) results in the hydrolysis of the silyl ester providing acid 8. Selective reduction of the carboxylic acid of 8 using, for example, borane-dimethylsulfide complex in a solvent (e.g., THF) provides an intermediate alcohol. This intermediate alcohol could be oxidized to aldehyde 9 by a number of known methods, including, but not limited to, Swern oxidation, Dess-Martin periodinane oxidation, Moffatt oxidation and the like.

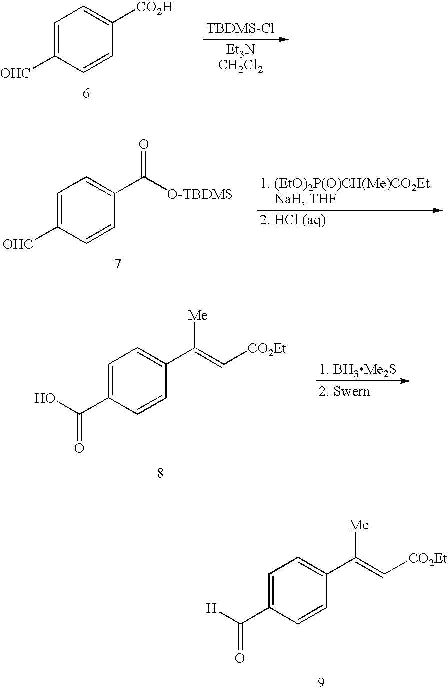

The aldehyde starting materials 2 or 9 can be reductively aminated to provide secondary or tertiary amines. This is illustrated by the reaction of methyl 4-formyl cinnamate 2 with tryptamine 10 using sodium triacetoxyborohydride (NaBH(OAc)₃) as the reducing agent in dichloroethane (DCE) as solvent to provide amine 11. Other reducing agents can be used, e.g., sodium borohydride (NaBH₄) and sodium cyanoborohydride (NaBH₃CN), in other solvents or solvent mixtures in the presence or absence of acid catalysts (e.g., acetic acid and trifluoroacetic acid). Amine 11 can be converted directly to hydroxamic acid 12 by treatment with 50% aqueous hydroxylamine in a suitable solvent (e.g., THF in the presence of a base, e.g., NaOH). Other methods of hydroxamate formation are known and include reaction of an ester with hydroxylamine hydrochloride and a base (e.g., sodium hydroxide or sodium methoxide) in a suitable solvent or solvent mixture (e.g., methanol, ethanol or methanol/THF).

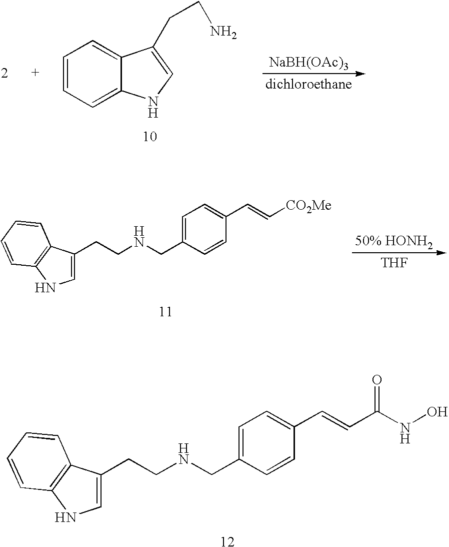

Aldehyde 2 can be reductively aminated with a variety of amines, exemplified by, but not limited to, those illustrated in Table 1. The resulting esters can be converted to target hydroxamates by the methods listed.

TABLE 1

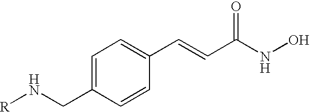

| | Reducing | Hydroxamate | |
|---|---|---|---|
| Amine | Conditions | Conditions | R |
| 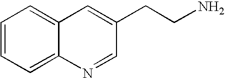 | NaBH(OAc)₃HOAc, DCE | 2 M HONH₂ inMeOH | 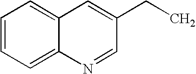 |
| 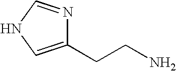 | NaBH(OAc)₃HOAc, DCE | 2 M HONH₂ inMeOH | 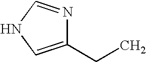 |
| 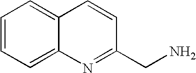 | NaBH(OAc)₃HOAc, DCE | 2 M HONH₂ inMeOH | 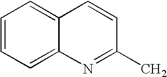 |
| 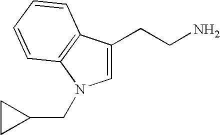 | NaBH(OAc)₃HOAc, DCE | 2 M HONH₂ inMeOH | 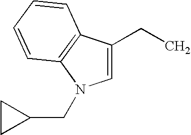 |
| 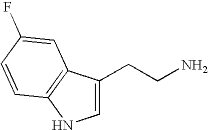 | NaBH(OAc)₃HOAc, DCE | 2 M HONH₂ inMeOH | 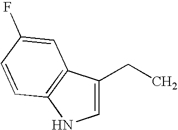 |
| 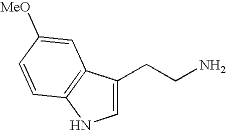 | NaBH(OAc)₃HOAc, DCE | 2 M HONH₂ inMeOH | 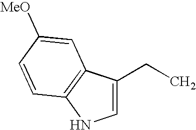 |
| 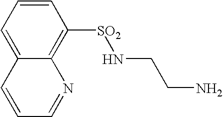 | NaBH(OAc)₃HOAc, DCE | 2 M HONH₂ inMeOH | 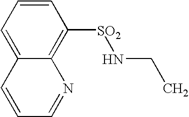 |
| 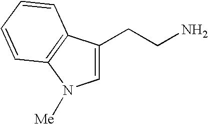 | NaBH(OAc)₃HOAc, DCE | 2 M HONH₂ inMeOH | 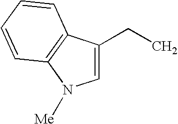 |
| 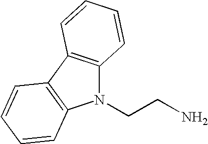 | NaBH(OAc)₃HOAc, DCE | 2 M HONH₂ inMeOH | 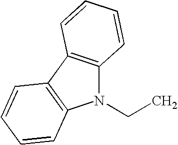 |
| Ph(CH₂)₃NH₂ | NaBH₃CN/MeOH/HOAc | | Ph(CH₂)₃ |

An alternate synthesis of the compounds of this invention starts by reductive amination of 4-formyl cinnamic acid 3, illustrated below with 3-phenylpropylamine 13, using, for example, NaBH₃CN as the reducing agent in MeOH and HOAc as a catalyst. The basic nitrogen of the resulting amino acid 14 can be protected, for example, as t-butoxycarbamate (BOC) by reaction with di-t-butyldicarbonate to give 15.

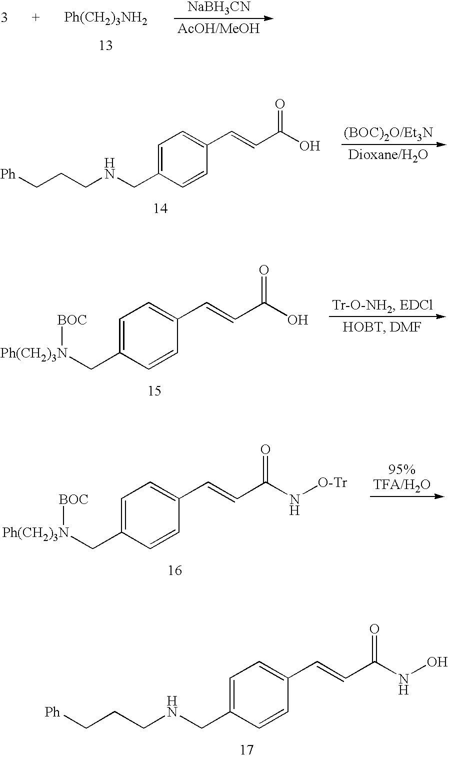

The carboxylic acid can be coupled with a protected hydroxylamine (e.g., O-trityl hydroxylamine) using a dehydrating agent (e.g., 1-(3-dimethylaminopropyl)-3-ethylcarbodiimide hydrochloride (EDCI)) and a catalyst (e.g., 1-hydroxybenzotriazole hydrate (HOBT)) in a suitable solvent (e.g., DMF) to produce 16. Treatment of 16 with a strong acid (e.g., trifluoroacetic acid (TFA)) provides a hydroxamic acid 17 of the present invention. Additional examples of compounds that can be prepared by this method are:

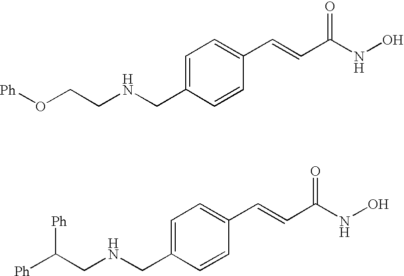

Tertiary amine compounds can be prepared by a number of methods. Reductive amination of 30 with nicotinaldehyde 32 using NaBH₃CN as the reducing agent in dichloroethane and HOAc as a catalyst provides ester 34. Other reducing agents can be used (e.g., NaBH₄ and NaBH(OAc)₃) in other solvents or solvent mixtures in the presence or absence of acid catalysts (e.g., acetic acid, trifluoroacetic acid and the like). Reaction of ester 34 with HONH₂.HCl, NaOH in MeOH provides hydroxamate 36.

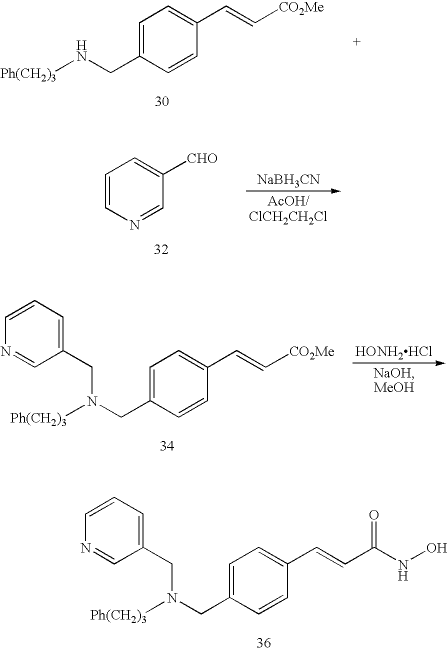

Tertiary amine compounds prepared by this methodology are exemplified, but not limited to, those listed in Table 2.

TABLE 2

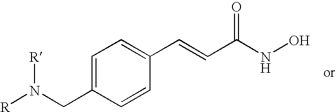

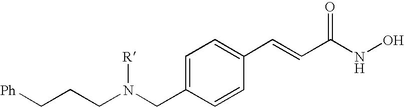

| | Reducing | Hydroxamate |
|---|---|---|
| | Conditions | Conditions |
| 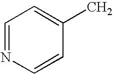 | NaBH(OAc)₃ HOAc,DCE | HONH₂•HCl/NaOMe/MeOH |
| 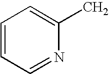 | NaBH(OAc)₃ HOAc,DCE | HONH₂•HCl/NaOMe/MeOH |
| 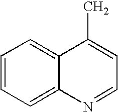 | NaBH(OAc)₃ HOAc,DCE | 2 M HONH₂ inMeOH |
| 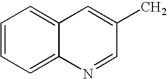 | NaBH(OAc)₃ HOAc,DCE | 2 M HONH₂ inMeOH |
| 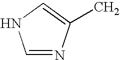 | NaBH₃CN/MeOH/HOAc | 2 M HONH₂ inMeOH |

An alternate method for preparing tertiary amines is by reacting a secondary amine with an alkylating agent in a suitable solvent in the presence of a base. For example, heating a dimethylsulfoxide (DMSO) solution of amine 11 and bromide 40 in the presence of (i-Pr)₂NEt yielded tertiary amine 42. Reaction of the tertiary amine 42 with HONH₂.HCl, NaOH in MeOH provides hydroxamate 43. The silyl group can be removed by any method known to those skilled in the art. For example, the hydroxamate 43 can be treated with an acid, e.g., trifluoroacetic acid, or fluoride to produce hydroxyethyl compound 44.

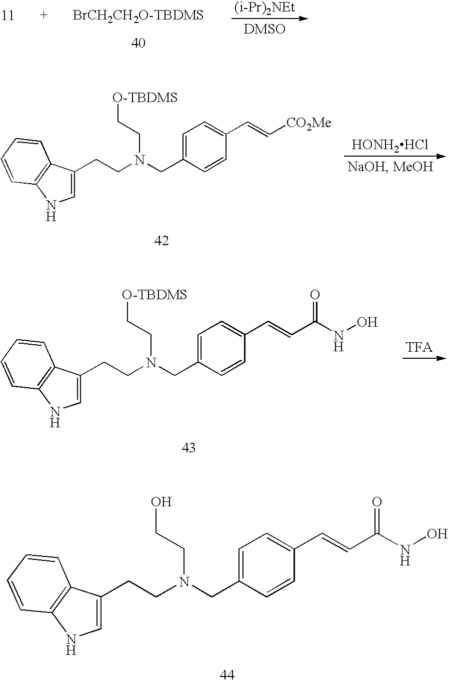

The hydroxamate compound, or salt thereof, is suitable for preparing pharmaceutical compositions, especially pharmaceutical compositions having deacetylase, especially histone deacetylase, inhibiting properties. Studies with athymic mice demonstrate that the hydroxamate compound causes HDA inhibition and increased histone acetylation in vivo, which triggers changes in gene expression that correlate with tumor growth inhibition.

The present invention further includes pharmaceutical compositions comprising a pharmaceutically effective amount of one or more of the above-described compounds as active ingredient. Pharmaceutical compositions according to the invention are suitable for enteral, such as oral or rectal, and parenteral administration to mammals, including man, for the treatment of tumors or pathological cardiac hypertrophy and heart failure, alone or in combination with one or more pharmaceutically acceptable carriers.

The hydroxamate compound is useful in the manufacture of pharmaceutical compositions having an effective amount the compound in conjunction or admixture with excipients or carriers suitable for either enteral or parenteral application. Preferred are tablets and gelatin capsules comprising the active ingredient together with (a) diluents; (b) lubricants, (c) binders (tablets); if desired, (d) disintegrants; and/or (e) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, the compositions may also contain other therapeutically valuable substances. The compositions are prepared according to conventional mixing, granulating or coating methods, respectively, and contain preferably about 1 to 50% of the active ingredient.

Suitable formulations also include formulations for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

In another embodiment, it is envisioned to use a hydroxamate compound in combination with other therapeutic modalities. Thus, in addition to the therapies described above, one may also provide to the patient more “standard” pharmaceutical cardiac therapies. Examples of standard therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 receptor antagonists and cytokine blockers/inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the hydroxamate compound therapy may precede or follow administration of the other agent by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

As discussed above, the compounds of the present invention are useful for treating and/or preventing a pathologically hypertrophied cardiac status and its adverse consequences including heart failure and arrhythmias. The inventive compounds are particularly useful for treating and/or preventing pathological cardiac hypertrophy including dilated cardiomyopathy and heart failure (diastolic, systolic, or combined diastolic and systolic) regardless of the precipitating event (e.g. myocardial infarction, etc.) or etiology (idiopathic, familial, drug-induced, or related to hypertension, valvular disease, ischemia, chronic alcoholism, infections, etc.).

The following examples are intended to illustrate the invention and are not to be construed as being limitations thereto.

### EXAMPLE P1

### Preparation of N-Hydroxy-3-[4-[[[2-(1H-indol-3-yl)-ethyl]-amino]methyl]phenyl]-2E-2-propenamide

4-formylcinnamic acid methylester is produced by adding 4-formylcinnamic acid (25 g, 0.143 mol) in MeOH and HCl (6.7 g, 0.18 mol). The resulting suspension is heated to reflux for 3 hours, cooled and evaporated to dryness. The resulting yellow solid is dissolved in EtOAc, the solution washed with saturated NaHCO₃, dried (MgSO₄) and evaporated to give a pale yellow solid which is used without further purification (25.0 g, 92%). To a solution of tryptamine (16.3 g, 100 mmol) and 4-formylcinnamic acid methylester (19 g, 100 mmol) in dichloroethane, NaBH(OAc)₃ (21 g, 100 mmol) is added. After 4 hours the mixture is diluted with 10% K₂CO₃ solution, the organic phase separated and the aqueous solution extracted with CH₂Cl₂. The combined organic extracts are dried (Na₂SO₄), evaporated and the residue purified by flash chromatography to produce 3-(4-{[2-(1H-indol-3-yl)-ethylamino]-methyl}-phenyl)-(2E)-2-propenoic acid methyl ester (29 g). A solution of KOH (12.9 g 87%, 0.2 mol) in MeOH (100 mL) is added to a solution of HONH₂.HCl (13.9 g, 0.2 mol) in MeOH (200 mL) and a precipitate results. After 15 minutes the mixture is filtered, the filter cake washed with MeOH and the filtrate evaporated under vacuum to approximately 75 mL. The mixture is filtered and the volume adjusted to 100 mL with MeOH. The resulting solution 2M HONH₂ is stored under N₂ at −20° C. for up to 2 weeks. Then 3-(4-{[2-(1H-indol-3-yl)-ethylamino]-methyl}-phenyl)-(2E)-2-propenoic acid methyl ester (2.20 g, 6.50 mmol) is added to 2 M HONH₂ in MeOH (30 mL, 60 mmol) followed by a solution of KOH (420 mg, 6.5 mmol) in MeOH (5 mL). After 2 hours dry ice is added to the reaction and the mixture is evaporated to dryness. The residue is dissolved in hot MeOH (20 mL), cooled and stored at −20° C. overnight. The resulting suspension is filtered, the solids washed with ice cold MeOH and dried under vacuum, producing N-Hydroxy-3-[4-[[[2-(1H-indol-3-yl)-ethyl]-amino]methyl]phenyl]-2E-2-propenamide (m/z 336 [MH⁺]).

### EXAMPLE P2

### Preparation of N-Hydroxy-3-[4-[[(2-hydroxyethyl)[2-(1H-indol-3-yl)-ethyl]-amino]methyl]phenyl]-2E-2-propenamide

A solution of 3-(4-{[2-(1H-indol-3-yl)-ethylamino]-methyl}-phenyl)-(2E)-2-propenoic acid methyl ester (12.6 g, 37.7 mmol), (2-bromoethoxy)-tert-butyldimethylsilane (12.8 g, 53.6 mmol), (i-Pr)₂NEt, (7.42 g, 57.4 mmol) in DMSO (100 mL) is heated to 50° C. After 8 hours the mixture is partitioned with CH₂Cl₂/H₂O. The organic layer is dried (Na₂SO₄) and evaporated. The residue is chromatographed on silica gel to produce 3-[4-({[2-(tert-butyldimethylsilanyloxy)-ethyl]-[2-(1H-indol-3-yl)-ethyl]-amino}-methyl)-phenyl]-(2E)-2-propenoic acid methyl ester (13.1 g). Following the procedure described for the preparation of the hydroxamate compound in Example P1, 3-[4-({[2-(tert-butyldimethylsilanyloxy)-ethyl]-[2-(1H-indol-3-yl)-ethyl]-amino}-methyl)-phenyl]-(2E)-2-propenoic acid methyl ester (5.4 g, 11 mmol) is converted to N-hydroxy-3-[4-({[2-(tert-butyldimethylsilanyloxy)-ethyl]-[2-(1H-indol-3-yl)-ethyl]-amino}-methyl)-phenyl]-(2E)-2-propenamide (5.1 g,) and used without further purification. The hydroxamic acid (5.0 g, 13.3 mmol) is then dissolved in 95% TFA/H₂O (59 mL) and heated to 40-50° C. for 4 hours. The mixture is evaporated and the residue purified by reverse phase HPLC to produce N-Hydroxy-3-[4-[[(2-hydroxyethyl)[2-(1H-indol-3-yl)-ethyl]-amino]methyl]phenyl]-2E-2-propenamide as the trifluoroacetate salt (m/z 380 [MH⁺]).

### EXAMPLE P3

### Preparation of N-hydroxy-3-[4-[[[2-(2-methyl-1H-indol-3-yl)-ethyl]-amino]methyl]phenyl]-2E-2-propenamide

A suspension of LiAlH₄ (17 g, 445 mmol) in dry THF (1000 mL) is cooled to 0° C. and 2-methylindole-3-glyoxylamide (30 g, 148 mmol) is added in portions over 30 min. The mixture is stirred at room temperature for 30 min. and then maintained at reflux for 3 h. The reaction is cooled to 0° C. and treated with H₂O (17 ml), 15% NaOH (aq., 17 ml) and H₂O (51 ml). The mixture is treated with MgSO₄, filtered and the filtrate evaporated to give 2-methyltryptamine which is dissolved in MeOH. Methyl 4-formylcinnamate (16.9 g, 88.8 mmol) is added to the solution, followed by NaBH₃CN (8.4 g) and AcOH (1 equiv.). After 1 h the reaction is diluted with NaHCO₃ (aq.) and extracted with EtOAc. The organic extracts are dried (MgSO₄), filtered and evaporated. The residue is purified by chromatography to give 3-(4-{[2-(2-methyl-1H-indol-3-yl)-ethylamino]-methyl}-phenyl)-(2E)-2-propenoic acid methyl ester. The ester is dissolved in MeOH, 1.0 M HCl/dioxane (1-1.5 eqiv.) is added followed by Et₂O. The resulting precipitate is filtered and the solid washed with Et₂O and dried thoroughly to give 3-(4-{[2-(2-methyl-1H-indol-3-yl)-ethylamino]-methyl}-phenyl)-(2E)-2-propenoic acid methyl ester hydrochloride. 1.0 M NaOH (aq., 85 mL) is added to an ice cold solution of the methyl ester hydrochloride (14.9 g, 38.6 mmol) and HONH₂ (50% aq. solution, 24.0 mL, ca. 391.2 mmol). After 6 h, the ice cold solution is diluted with H₂O and NH₄Cl (aq., 0.86 M, 100 mL). The resulting precipitate is filtered, washed with H₂O and dried to afford N-hydroxy-3-[4-[[[2-(2-methyl-1H-indol-3-yl)-ethyl]-amino]methyl]phenyl]-2E-2-propenamide (m/z 350 [MH⁺]).

### EXAMPLES 1-265

The following compounds are prepared by methods analogous to those disclosed in Examples P1, P2 and P3:

| | | m/z |
|---|---|---|
| Example | STRUCTURE | (MH⁺) |
| 1 | 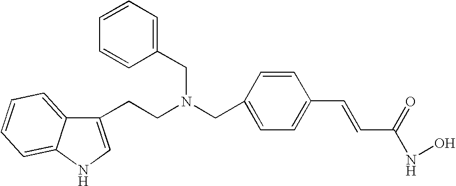 | 426 |
| 2 | 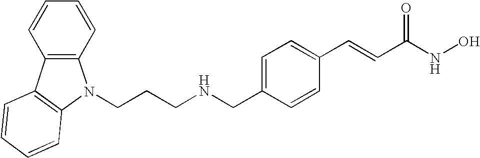 | |
| 3 | 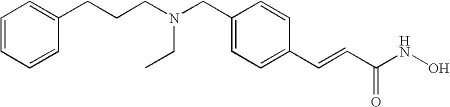 | |
| 4 | 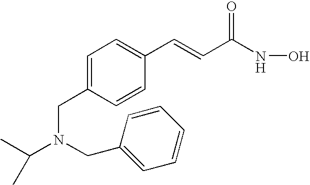 | 325 |
| 5 | 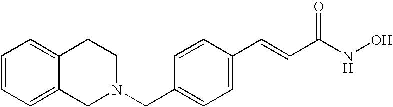 | |
| 6 | 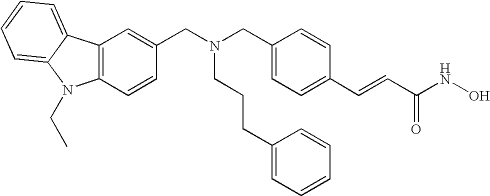 | |
| 7 | 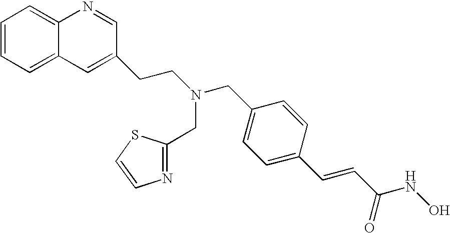 | |
| 8 | 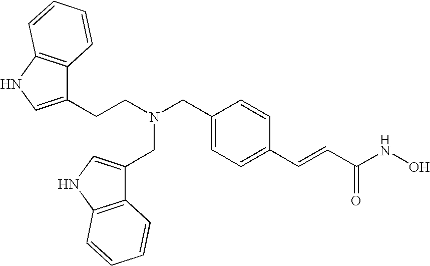 | |
| 9 | 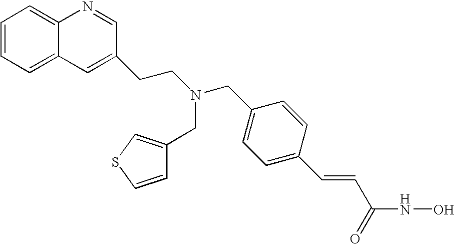 | |
| 10 | 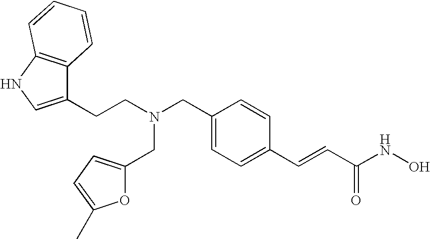 | |
| 11 | 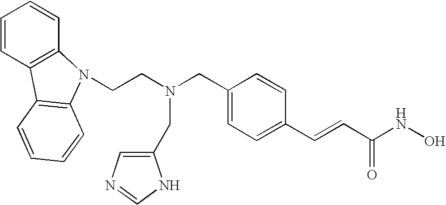 | |
| 12 | 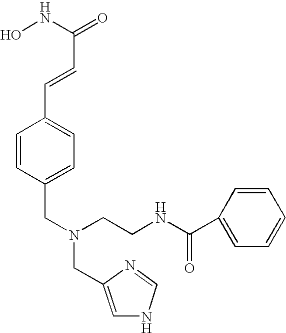 | 420 |
| 13 | 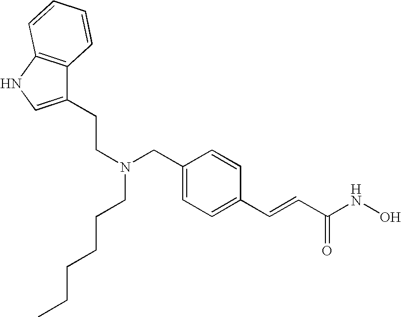 | 420 |
| 14 | 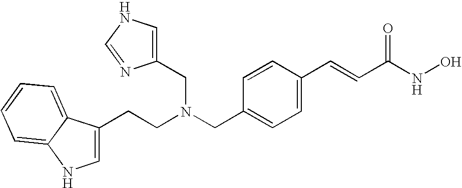 | |
| 15 | 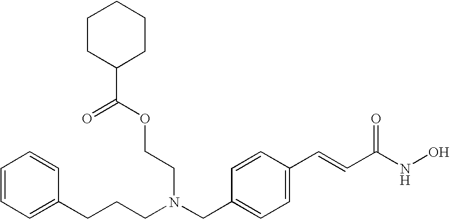 | 465 |
| 16 | 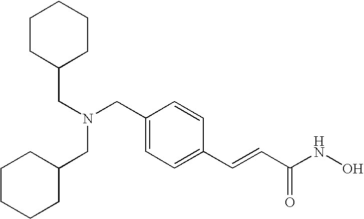 | 385 |
| 17 | 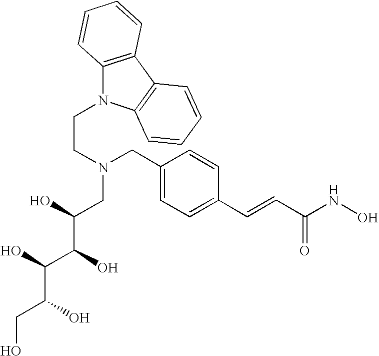 | 550 |
| 18 | 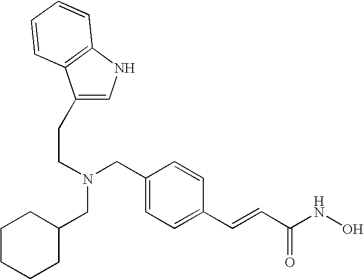 | 432 |
| 19 | 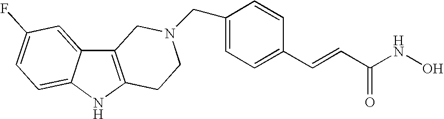 | 366 |
| 20 | 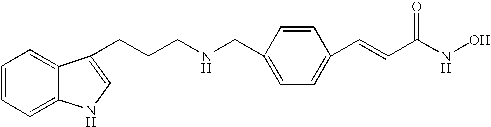 | 350 |
| 21 | 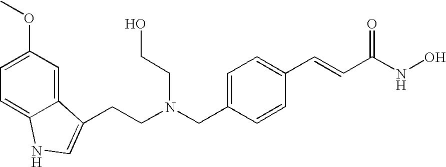 | |
| 22 | 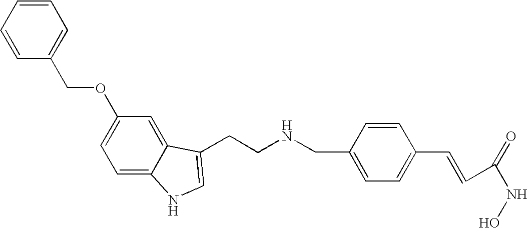 | 442 |
| 23 | 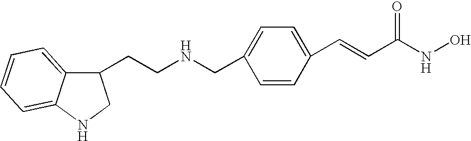 | 338 |
| 24 | 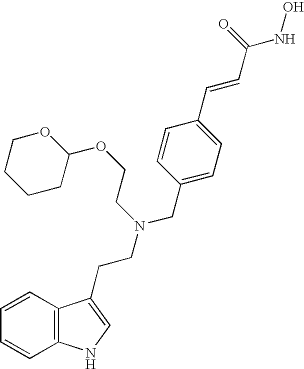 | 464 |
| 25 | 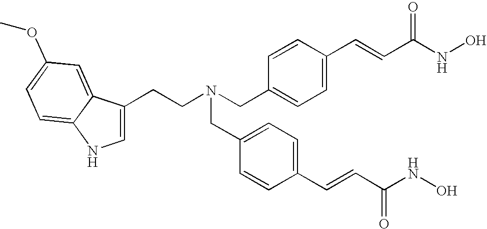 | 541 |
| 26 | 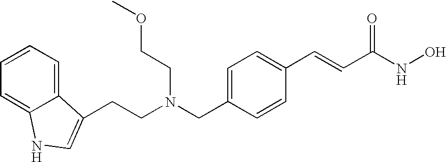 | |
| 27 | 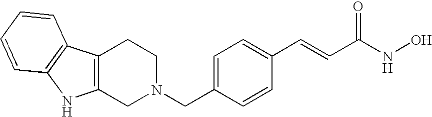 | |
| 28 | 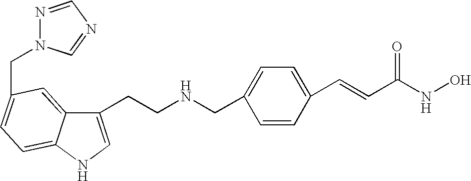 | 417 |
| 29 | 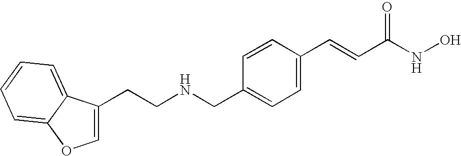 | |
| 30 | 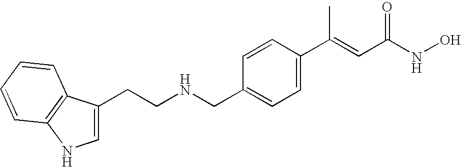 | |
| 31 | 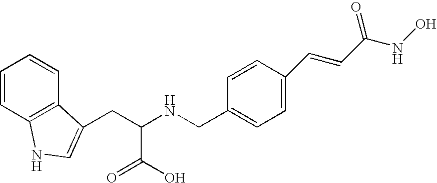 | 380 |
| 32 | 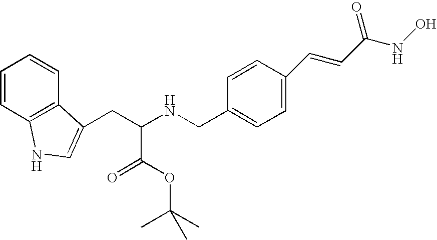 | 436 |
| 33 | 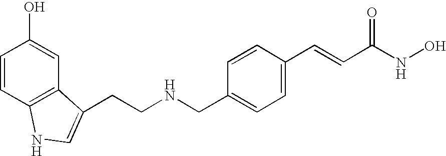 | |
| 34 | 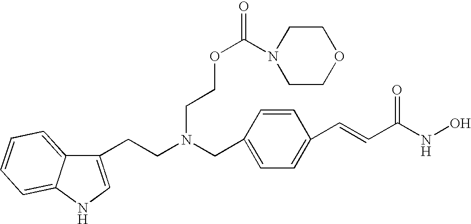 | 493 |
| 35 | 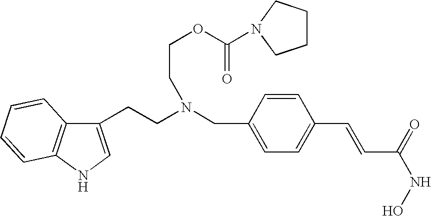 | 477 |
| 36 | 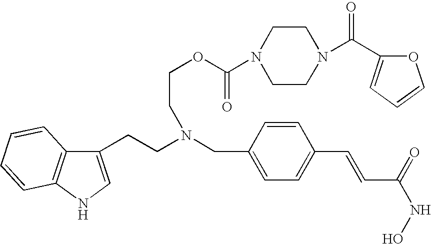 | 586 |
| 37 | 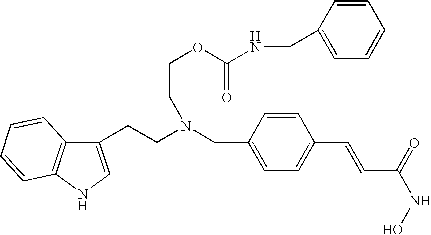 | 513 |
| 38 | 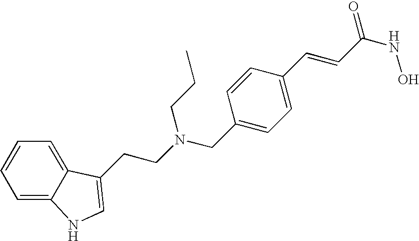 | 378 |
| 39 | 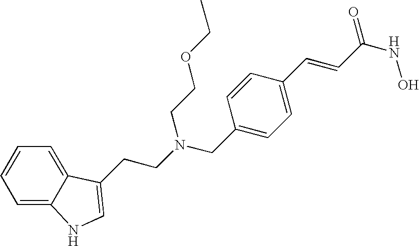 | 408 |
| 40 | 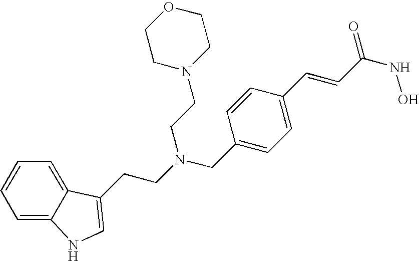 | 449 |
| 41 | 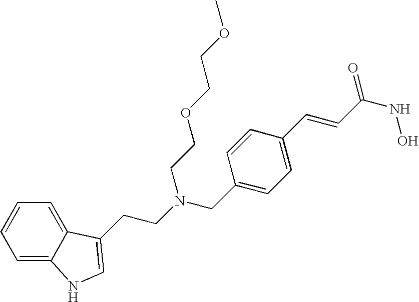 | 438 |
| 42 | 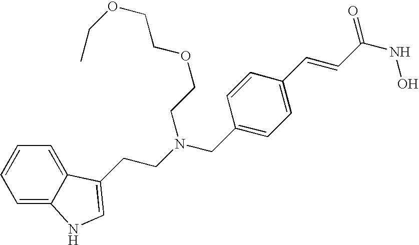 | 452 |
| 43 | 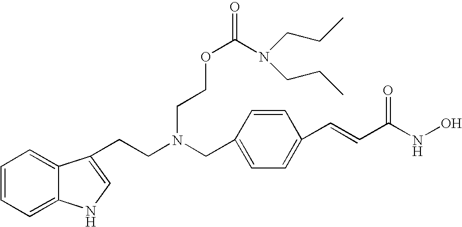 | 507 |
| 44 | 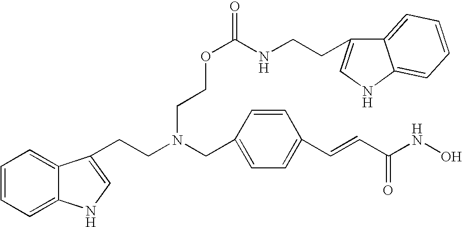 | 565 |
| 45 | 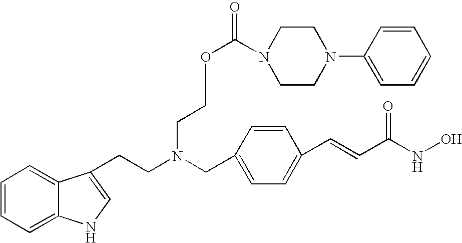 | |
| 46 | 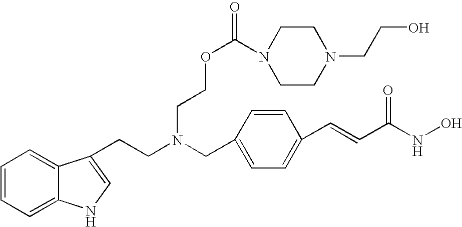 | |
| 47 | 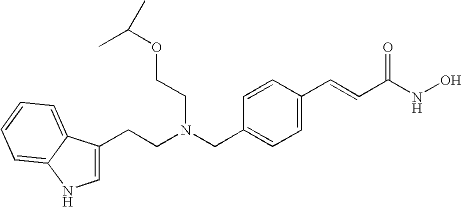 | |
| 48 | 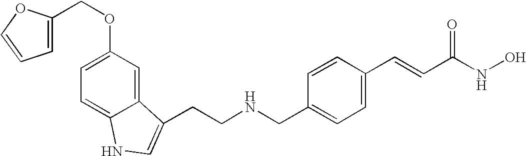 | |
| 49 | 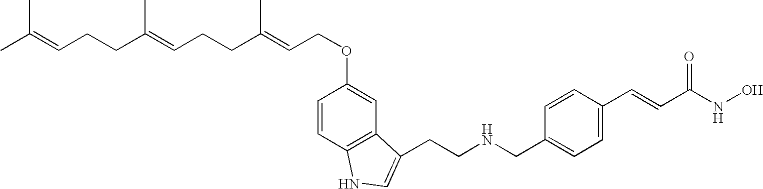 | |
| 50 | 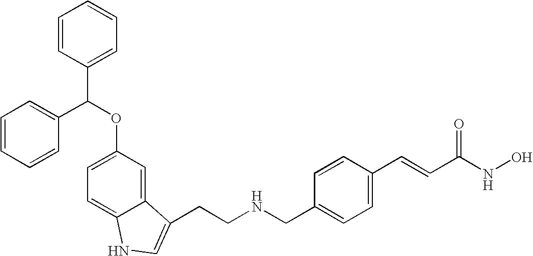 | |
| 51 | 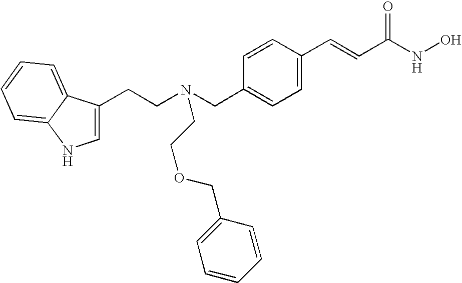 | 470 |
| 52 | 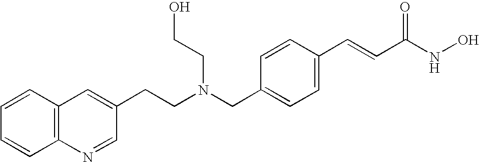 | |
| 53 | 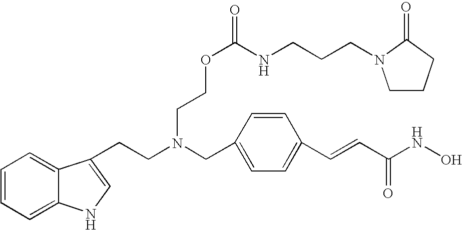 | 548 |
| 54 | 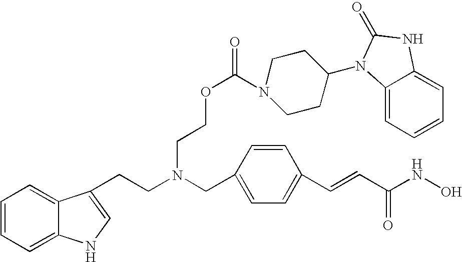 | 623 |
| 55 | 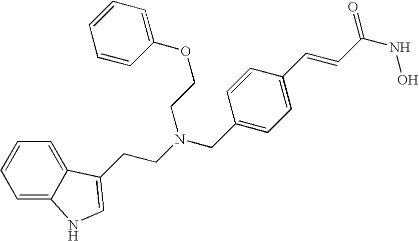 | 456 |
| 56 | 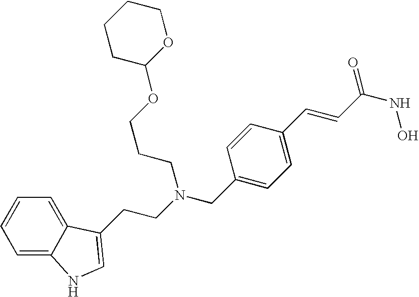 | 478 |
| 57 | 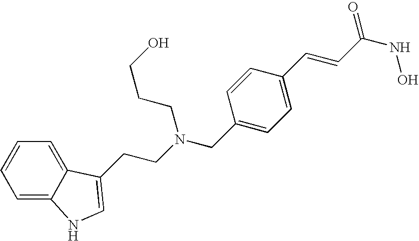 | 394 |
| 58 | 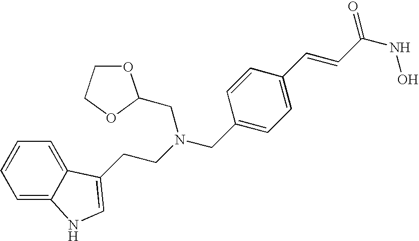 | 422 |
| 59 | 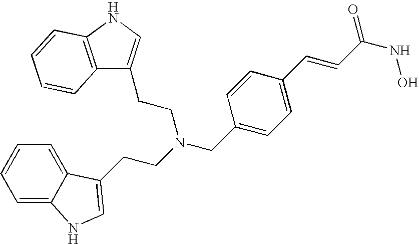 | 479 |
| 60 | 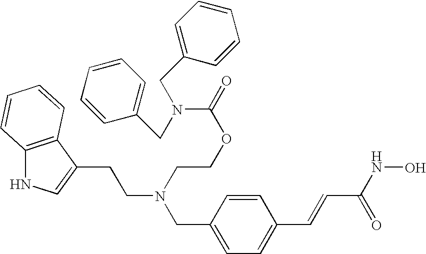 | 603 |
| 61 | 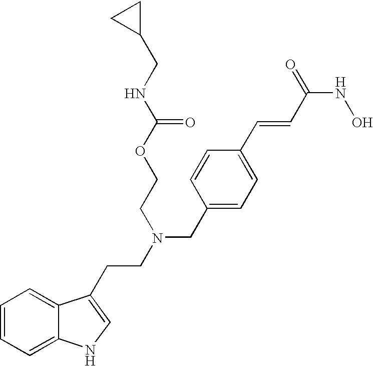 | 477 |
| 62 | 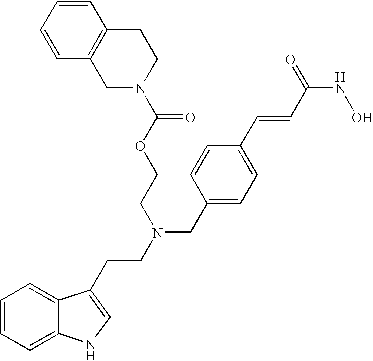 | 539 |
| 63 | 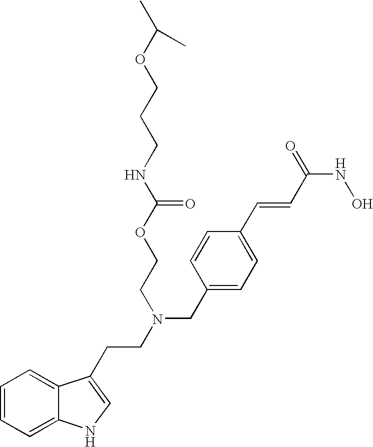 | 523 |
| 64 | 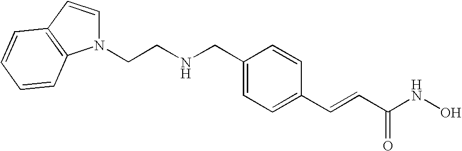 | |
| 65 | 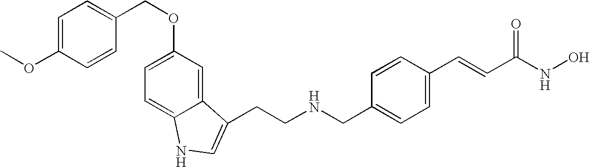 | |
| 66 | 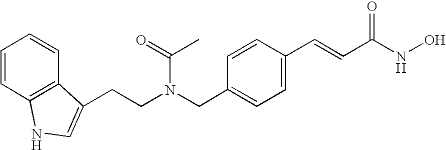 | |
| 67 | 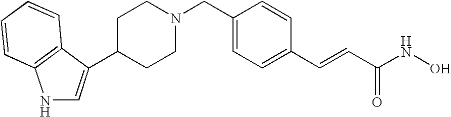 | |
| 68 | 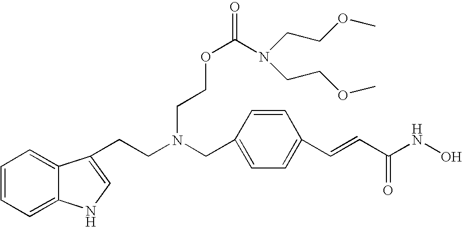 | 539 |
| 69 | 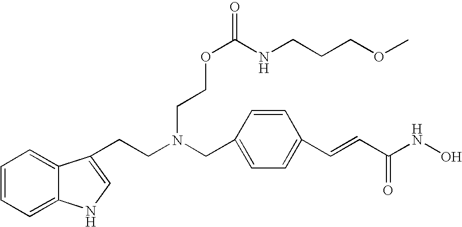 | 495 |
| 70 | 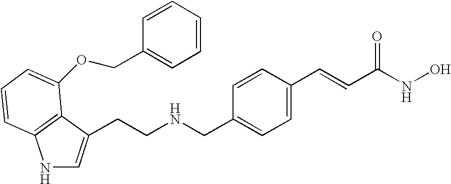 | |
| 71 | 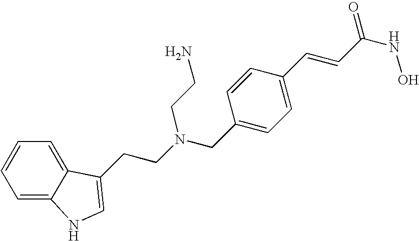 | 379 |
| 72 | 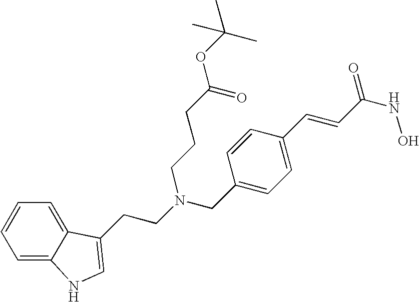 | 478 |
| 73 | 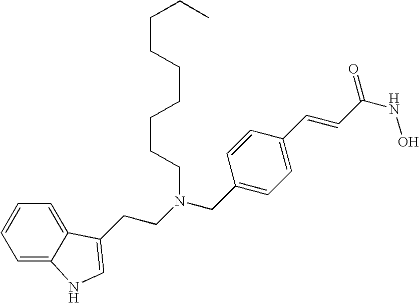 | 462 |
| 74 | 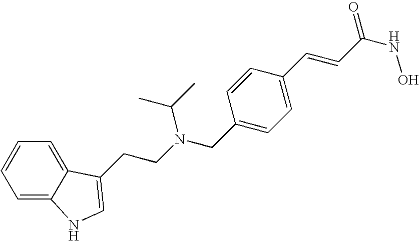 | 378 |
| 75 | 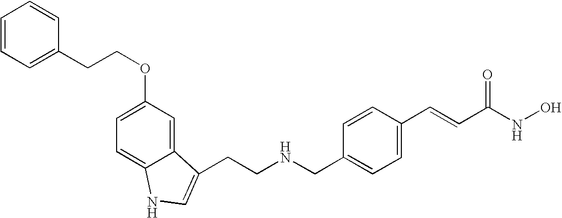 | |
| 76 | 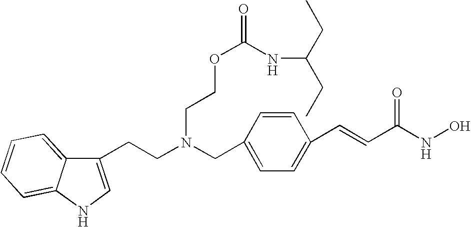 | 493 |
| 77 | 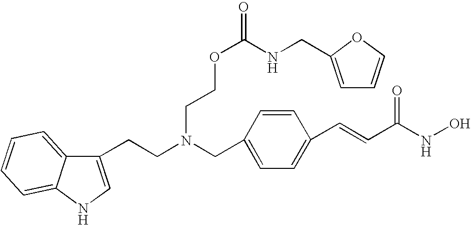 | 503 |
| 78 | 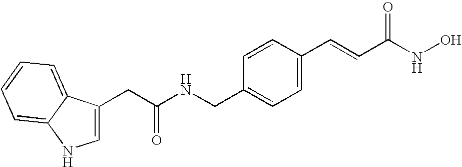 | 350 |
| 79 | 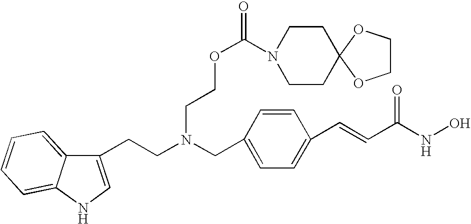 | 549 |
| 80 | 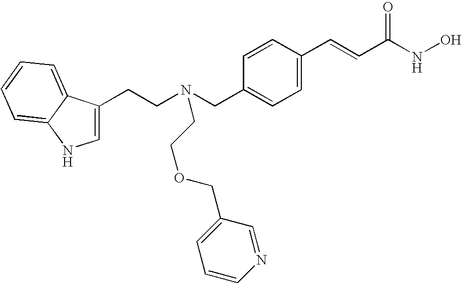 | 471 |
| 81 | 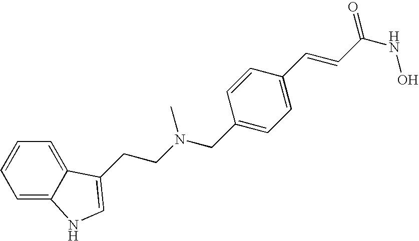 | 350 |
| 82 | 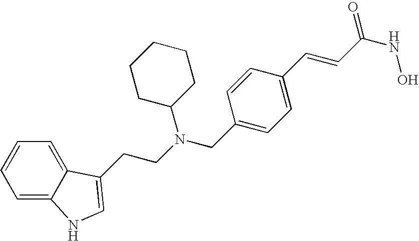 | 418 |
| 83 | 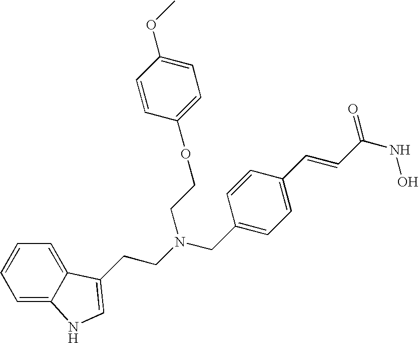 | 486 |
| 84 | 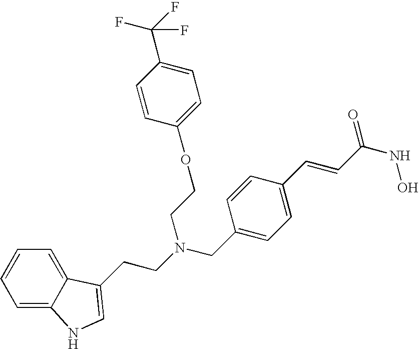 | 524 |
| 85 | 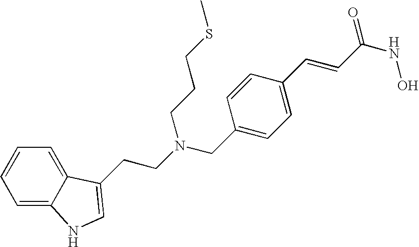 | 424 |
| 86 | 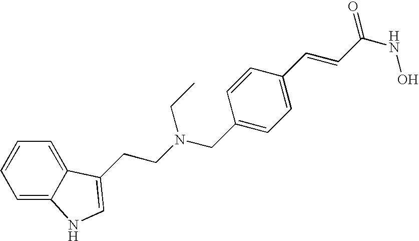 | 364 |
| 87 | 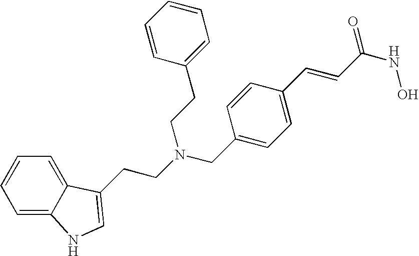 | 440 |
| 88 | 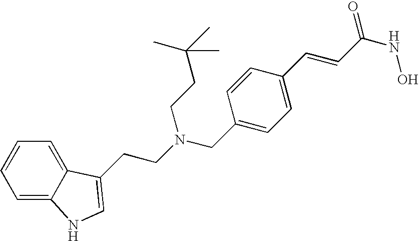 | 420 |
| 89 | 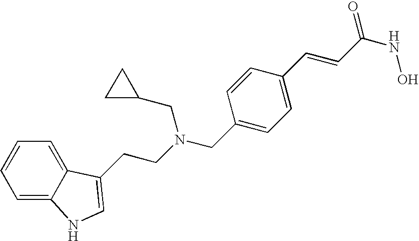 | 390 |
| 90 | 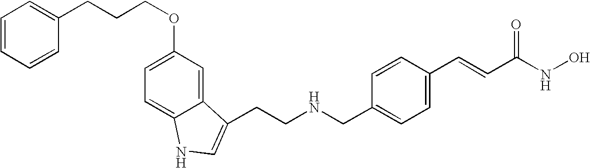 | |
| 91 | 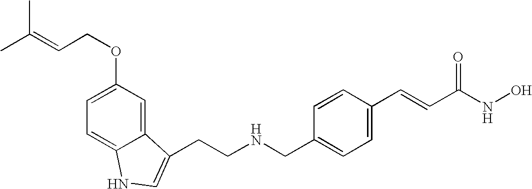 | |
| 92 | 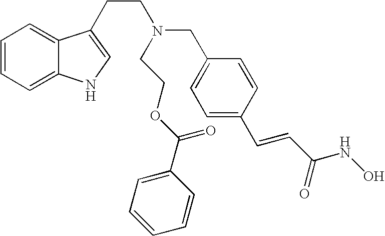 | 484 |
| 93 | 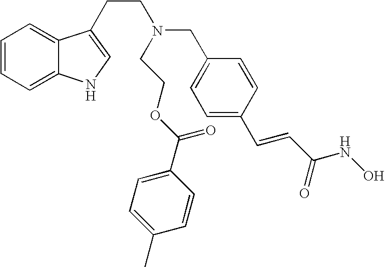 | 498 |
| 94 | 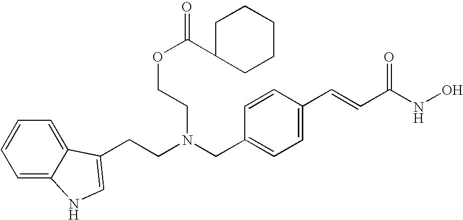 | 490 |
| 95 | 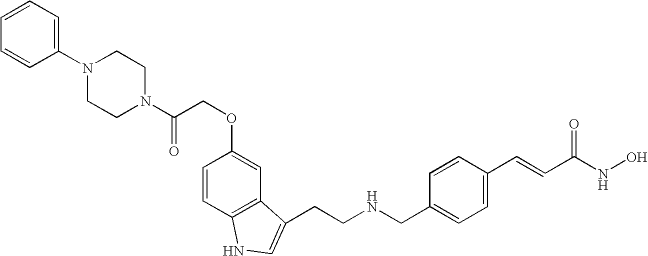 | |
| 96 | 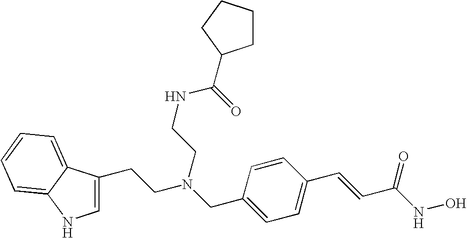 | 475 |
| 97 | 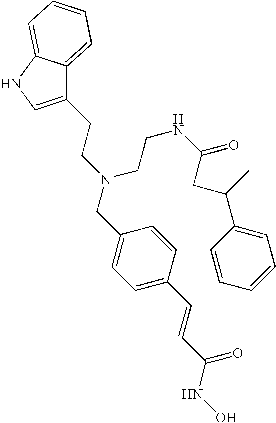 | 525 |
| 98 | 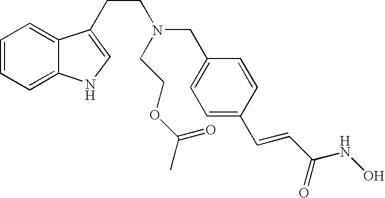 | 422 |
| 99 | 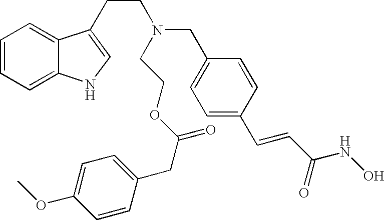 | 528 |
| 100 | 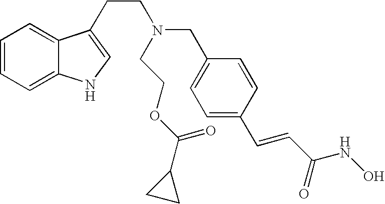 | 448 |
| 101 | 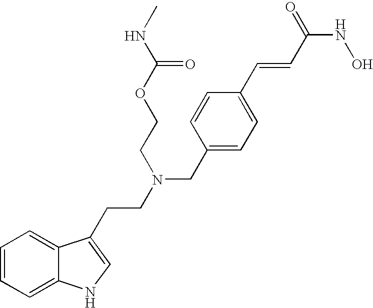 | 437 |
| 102 | 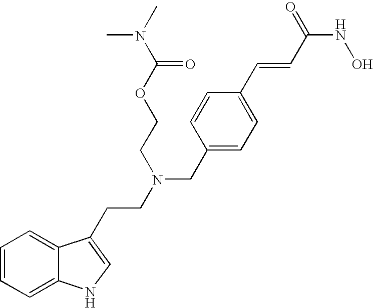 | 451 |
| 103 | 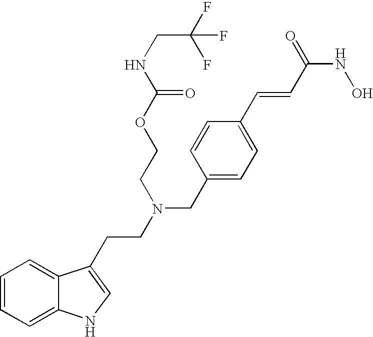 | 505 |
| 104 | 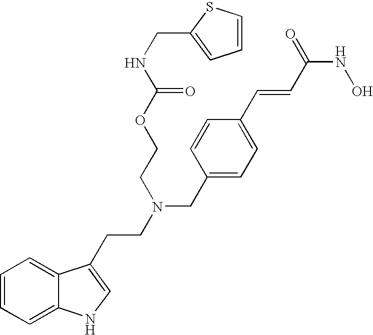 | 519 |
| 105 | 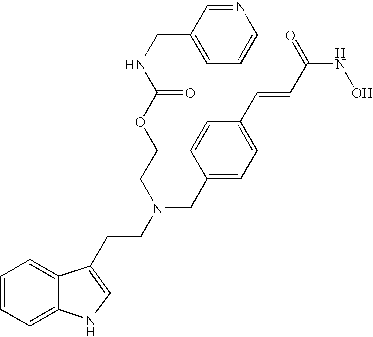 | 514 |
| 106 | 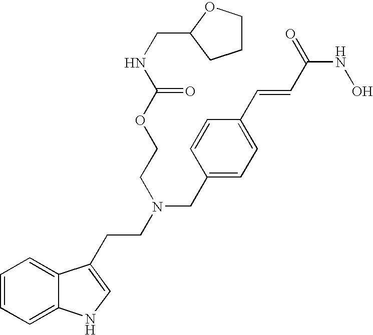 | 507 |
| 107 | 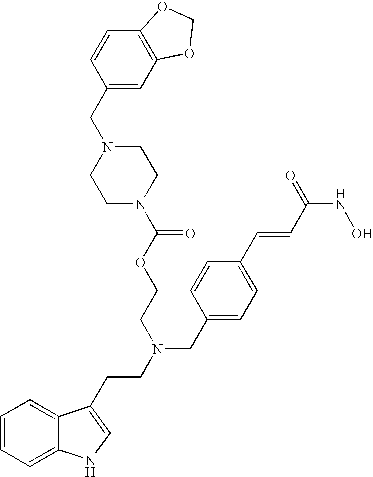 | 626 |
| 108 | 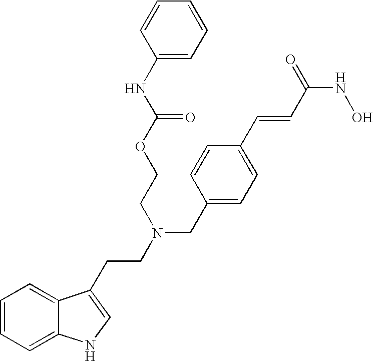 | 499 |
| 109 | 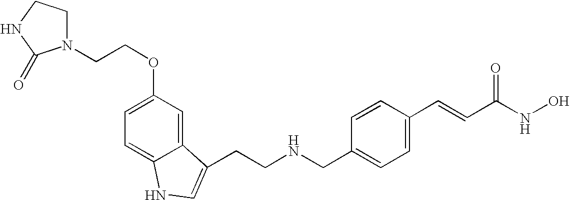 | |
| 110 | 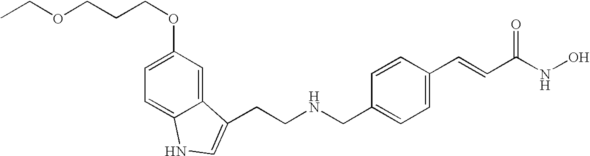 | |
| 111 | 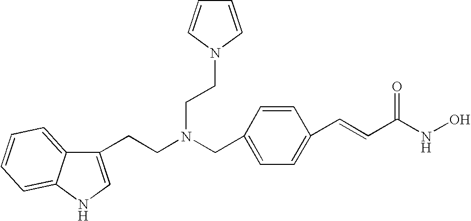 | 429 |
| 112 | 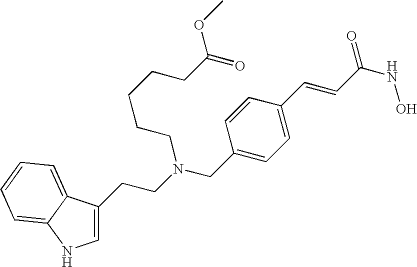 | 464 |
| 113 | 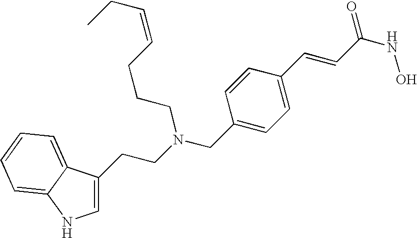 | 432 |
| 114 | 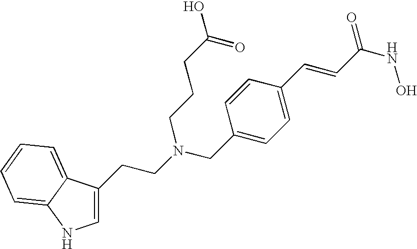 | 422 |
| 115 | 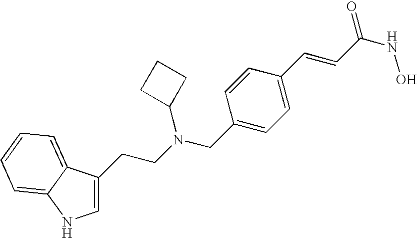 | 390 |
| 116 | 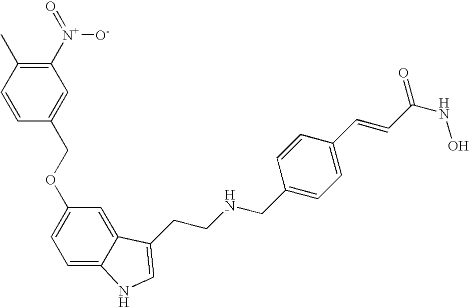 | 501 |
| 117 | 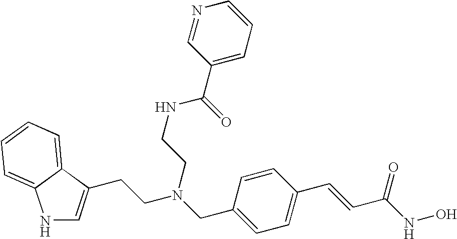 | 484 |
| 118 | 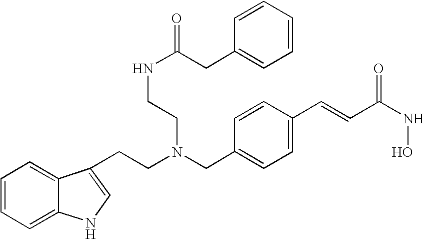 | |
| 119 | 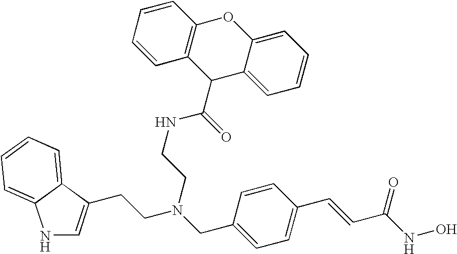 | 587 |
| 120 | 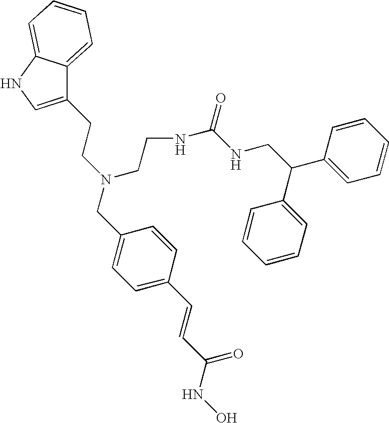 | 602 |
| 121 | 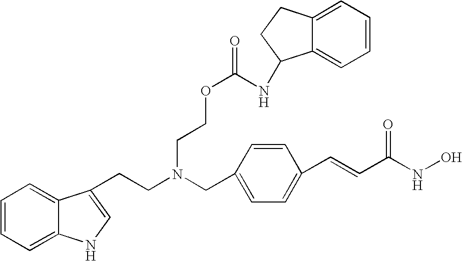 | 539 |
| 122 | 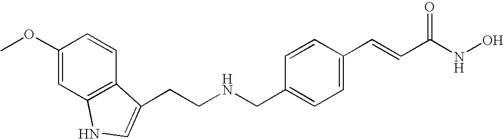 | |
| 123 | 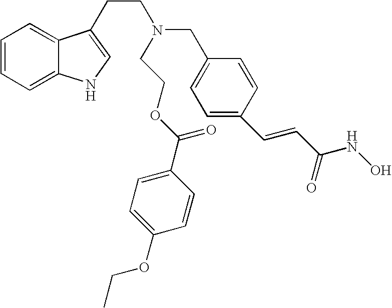 | 528 |
| 124 | 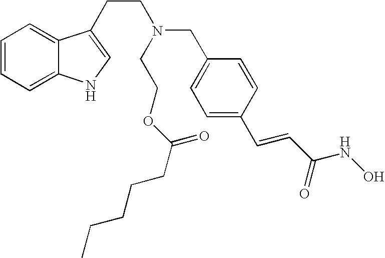 | 487 |
| 125 | 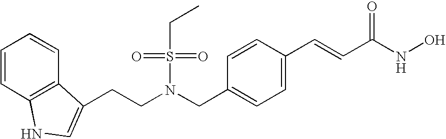 | |
| 126 | 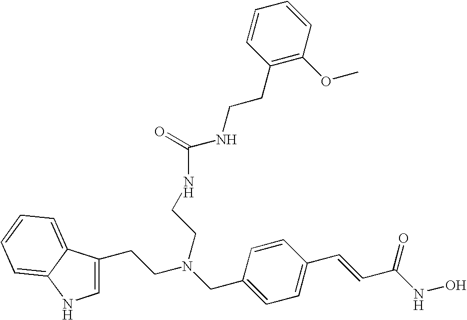 | 556 |
| 127 | 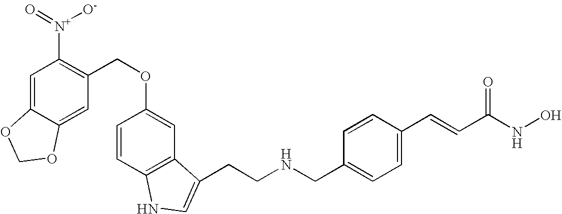 | |
| 128 | 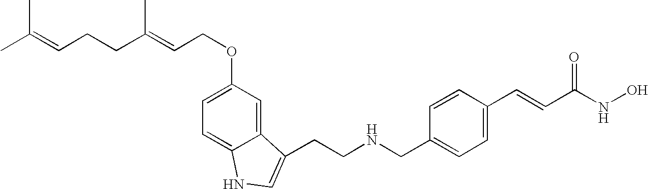 | |
| 129 | 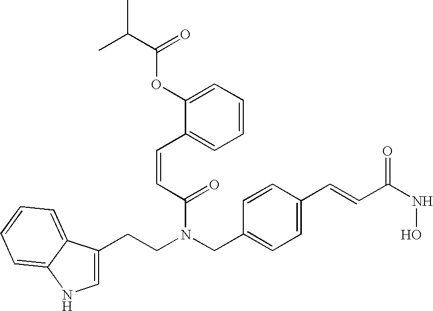 | 552 |
| 130 | 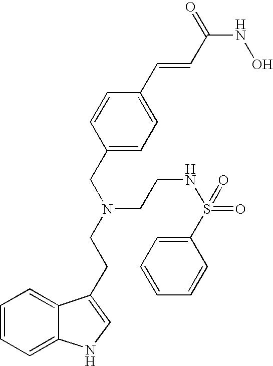 | 519 |
| 131 | 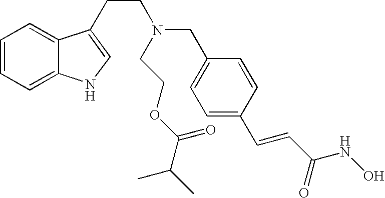 | 450 |
| 132 | 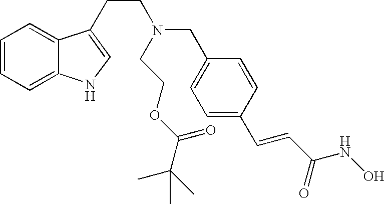 | 464 |
| 133 | 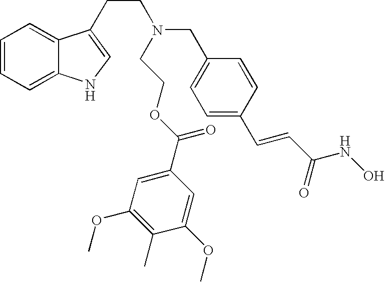 | 558 |
| 134 | 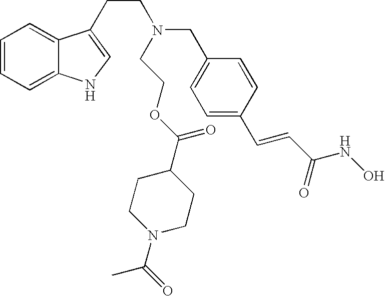 | 533 |
| 135 | 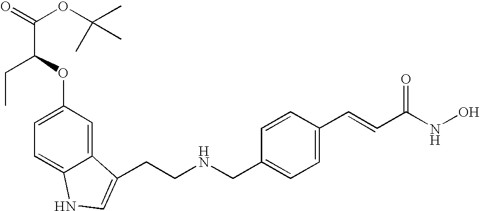 | |
| 136 | 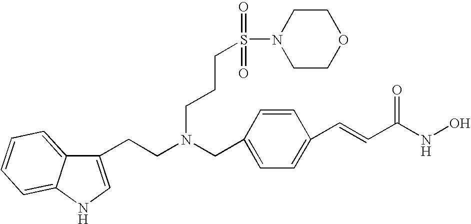 | 527 |
| 137 | 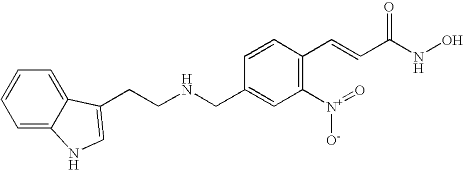 | 381 |
| 138 | 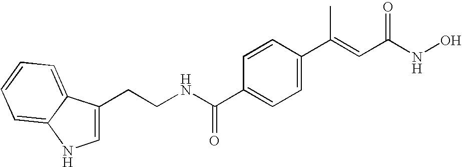 | 364 |
| 139 | 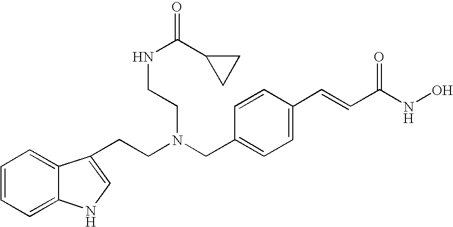 | |
| 140 | 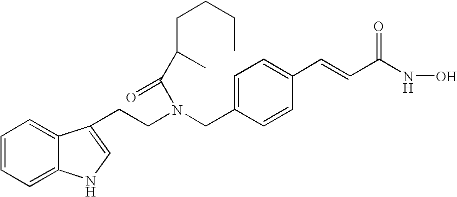 | 448 |
| 141 | 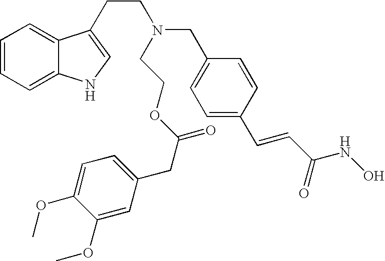 | 558 |
| 142 | 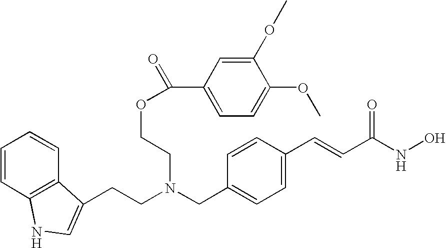 | |
| 143 | 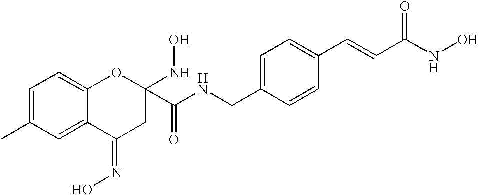 | 427 |
| 144 | 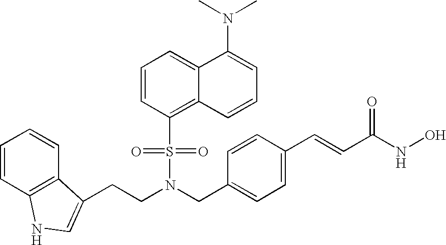 | |
| 145 | 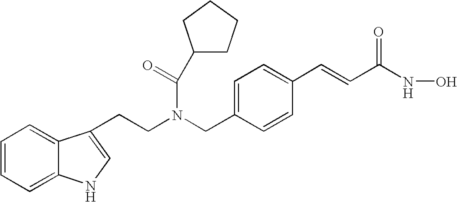 | 432 |
| 146 | 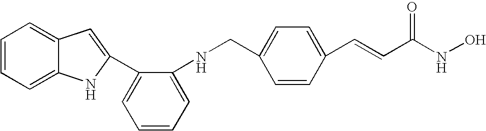 | 384 |
| 147 | 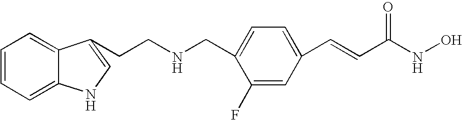 | 354 |
| 148 | 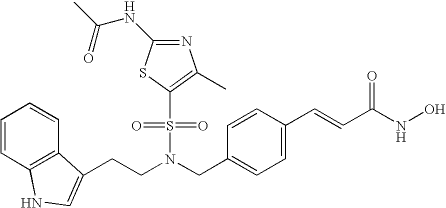 | |
| 149 | 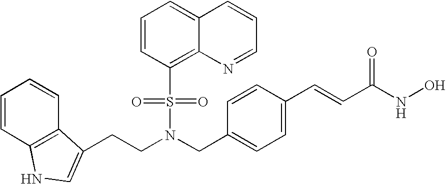 | |
| 150 | 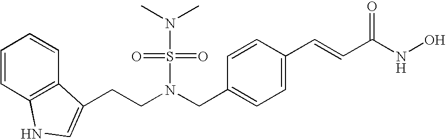 | |
| 151 | 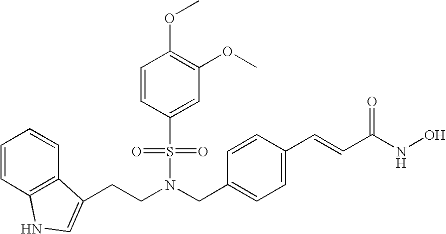 | |
| 152 | 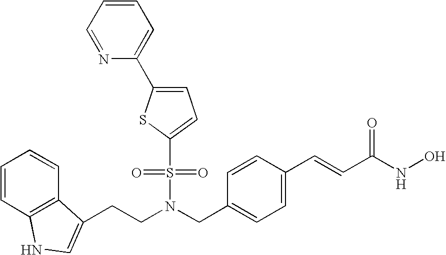 | |
| 153 | 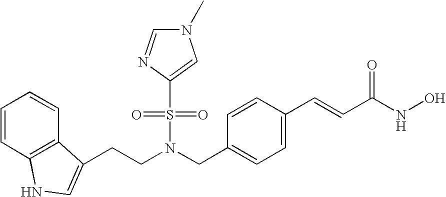 | |
| 154 | 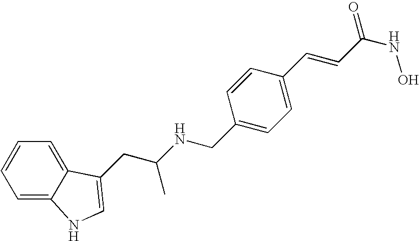 | 350 |
| 155 | 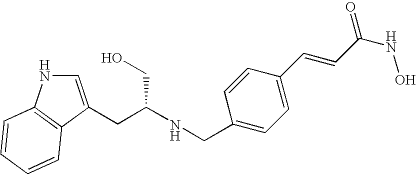 | 366 |
| 156 | 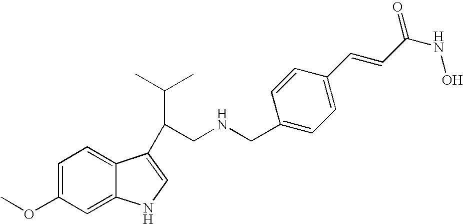 | 408 |
| 157 | 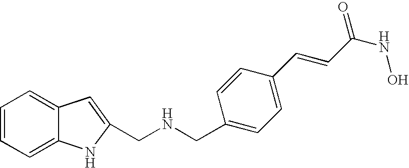 | 322 |
| 158 | 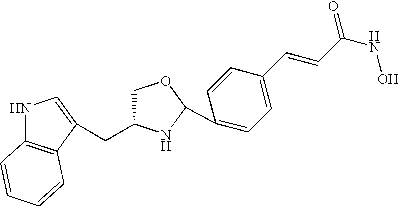 | 364 |
| 159 | 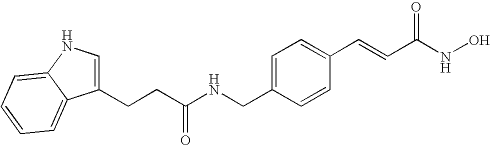 | 364 |
| 160 | 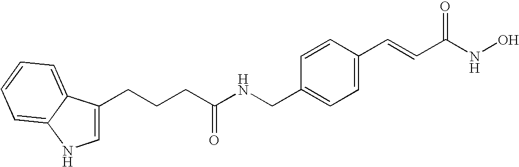 | 378 |
| 161 | 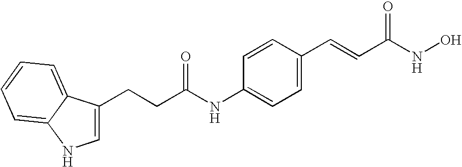 | 350 |
| 162 | 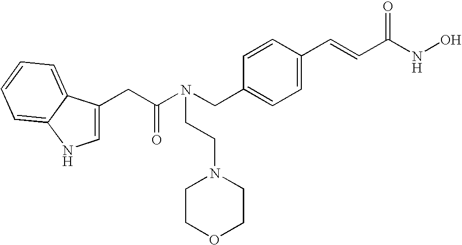 | 463 |
| 163 | 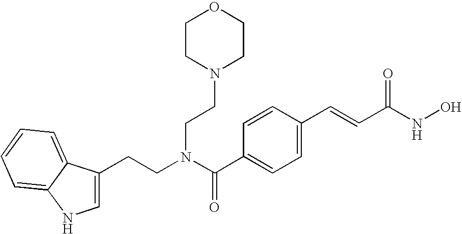 | |
| 164 | 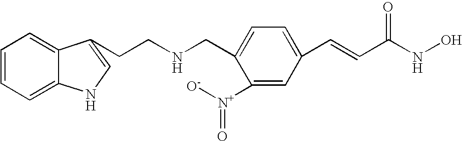 | 381 |
| 165 | 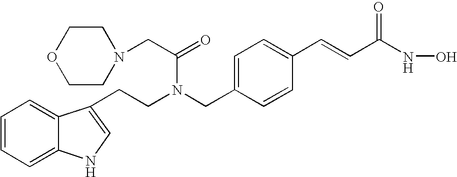 | 463 |
| 166 | 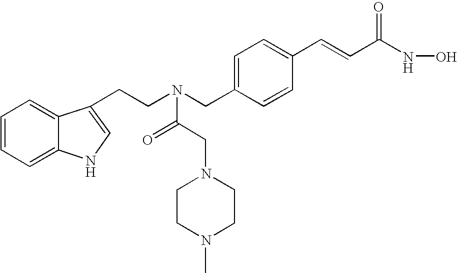 | 476 |
| 167 | 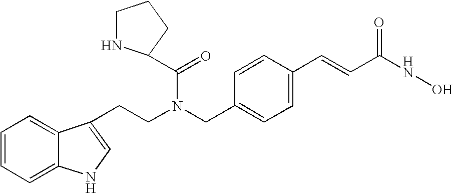 | |
| 168 | 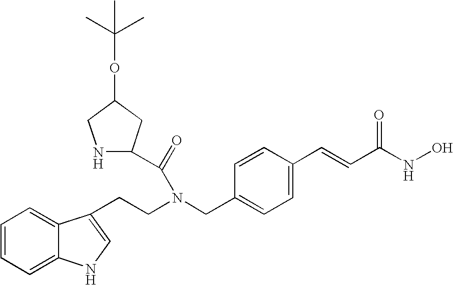 | |
| 169 | 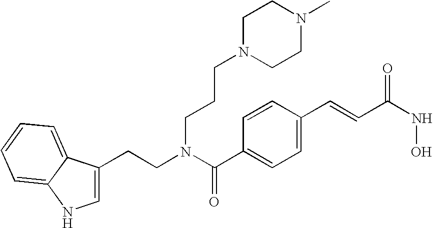 | |
| 170 | 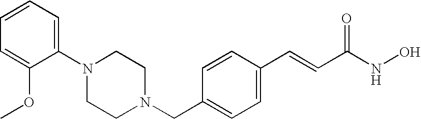 | 368 |
| 171 | 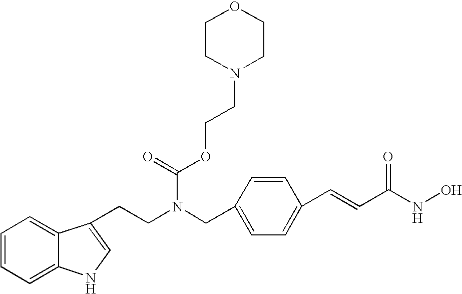 | 493 |
| 172 | 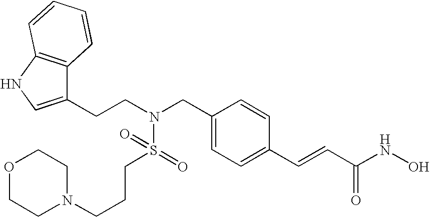 | 527 |
| 173 | 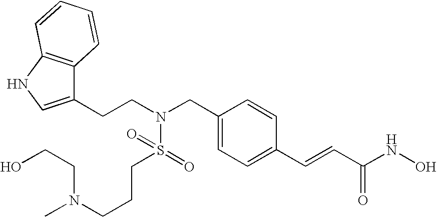 | 515 |
| 174 | 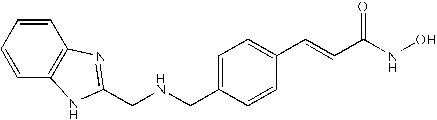 | 323 |
| 175 | 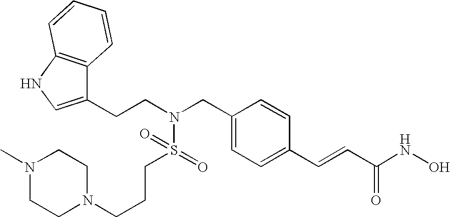 | 540 |
| 176 | 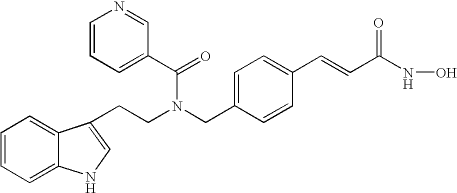 | 441 |
| 177 | 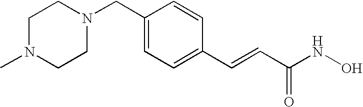 | 276 |
| 178 | 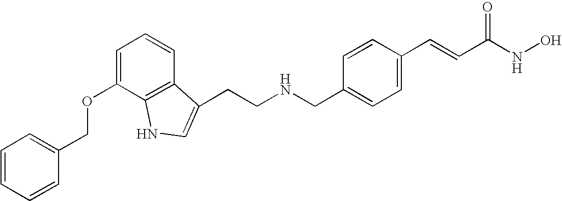 | |
| 179 | 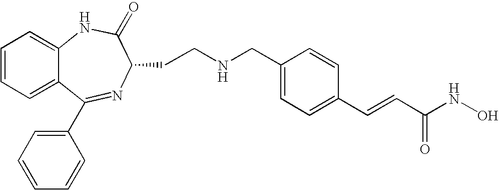 | 455 |
| 180 | 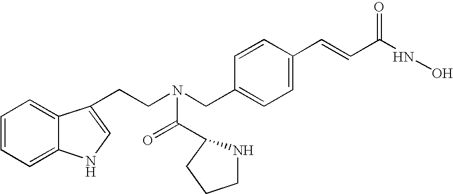 | |
| 181 | 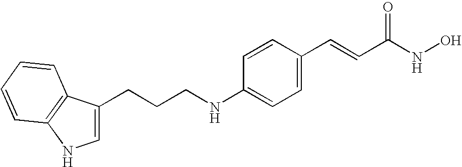 | 336 |
| 182 | 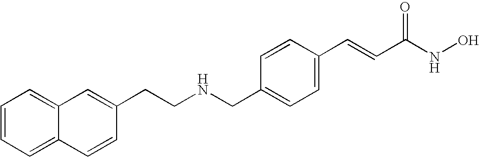 | 347 |
| 183 | 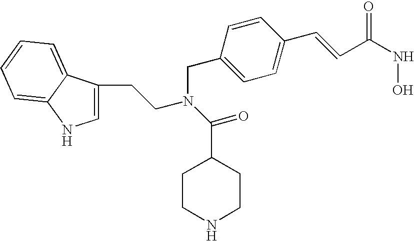 | 447 |
| 184 | 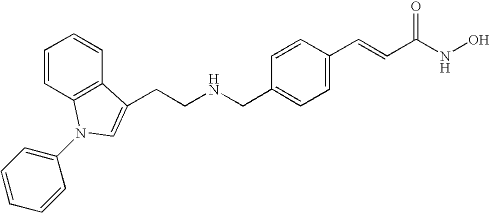 | |
| 185 | 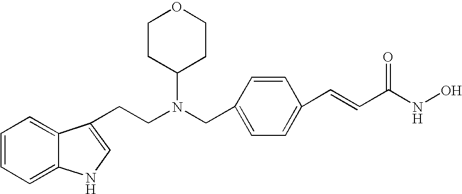 | 420 |
| 186 | 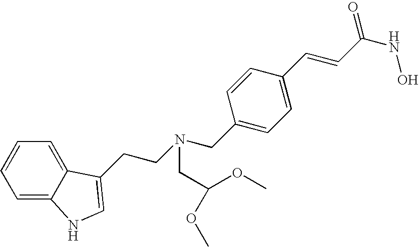 | 424 |
| 187 | 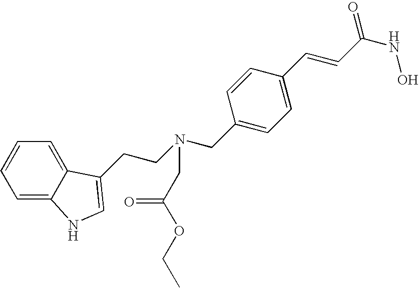 | 422 |
| 188 | 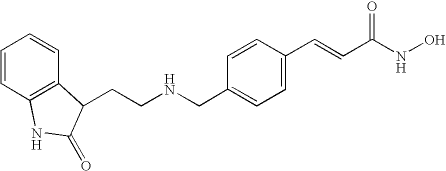 | |
| 189 | 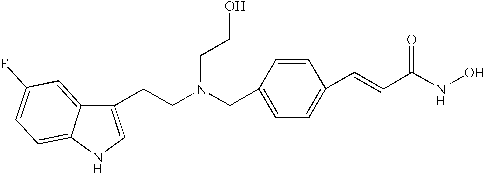 | 398 |
| 190 | 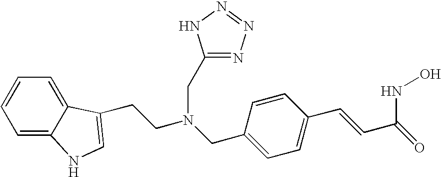 | 418 |
| 191 | 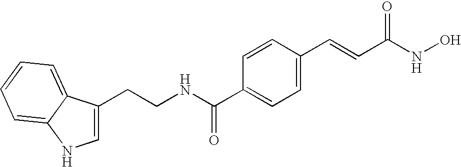 | 350 |
| 192 | 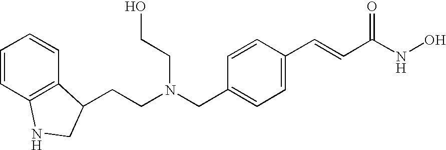 | |
| 193 | 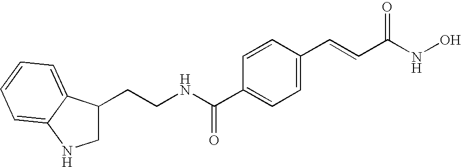 | 352 |
| 194 | 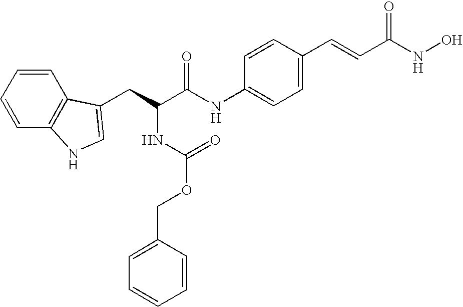 | 499 |
| 195 | 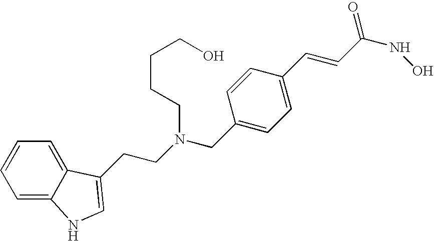 | 408 |
| 196 | 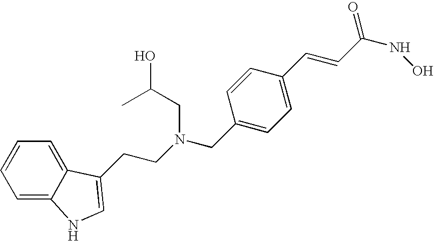 | 394 |
| 197 | 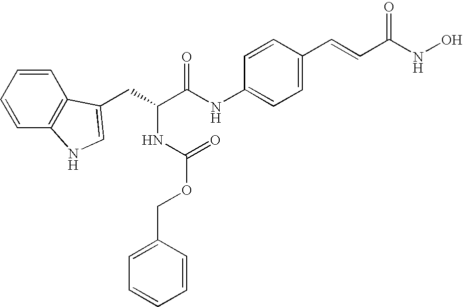 | 499 |
| 198 | 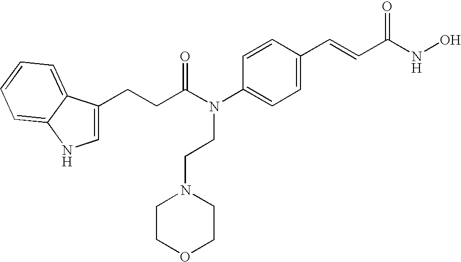 | |
| 199 | 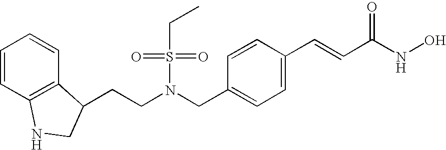 | |
| 200 | 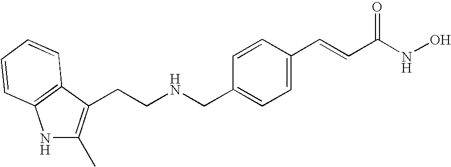 | 350 |
| 201 | 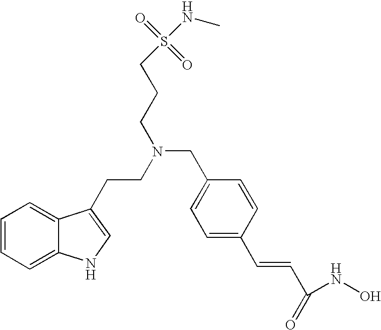 | |
| 202 | 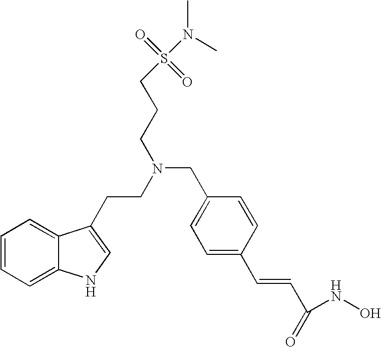 | |
| 203 | 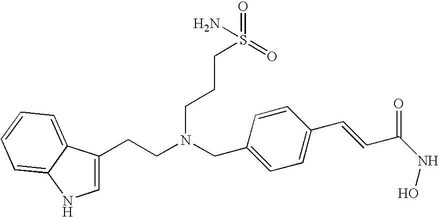 | |
| 204 | 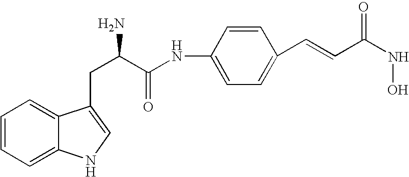 | 365 |
| 205 | 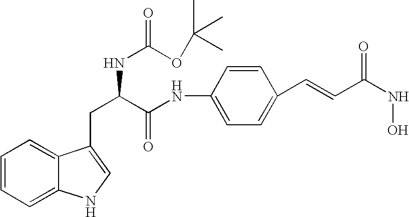 | 465 |
| 206 | 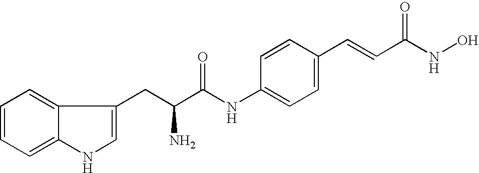 | |
| 207 | 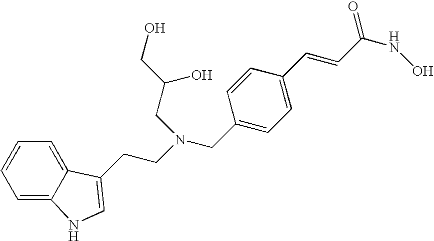 | 410 |
| 208 | 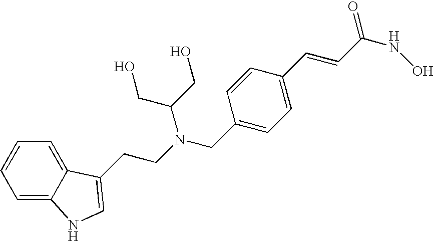 | 410 |
| 209 | 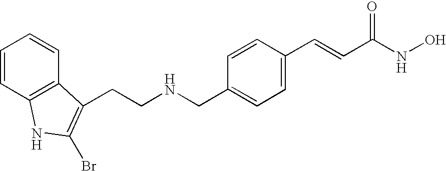 | |
| 210 | 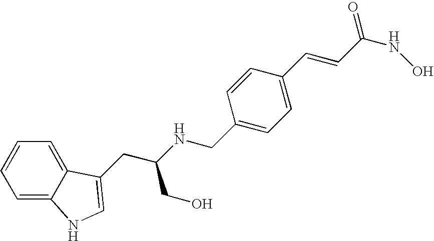 | 366 |
| 211 | 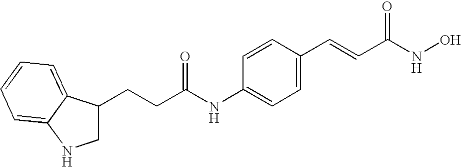 | 352 |
| 212 | 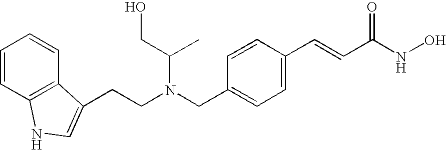 | |
| 213 | 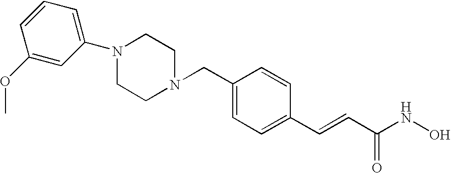 | 368 |
| 214 | 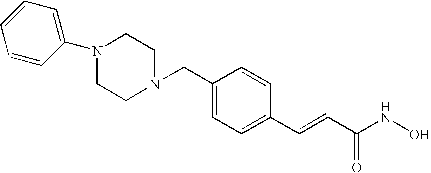 | 338 |
| 215 | 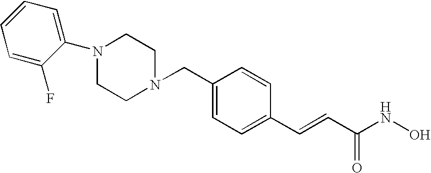 | 356 |
| 216 | 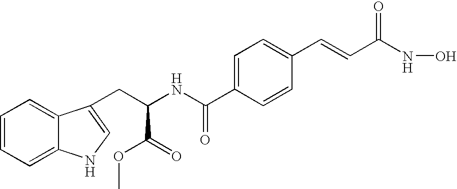 | 408 |
| 217 | 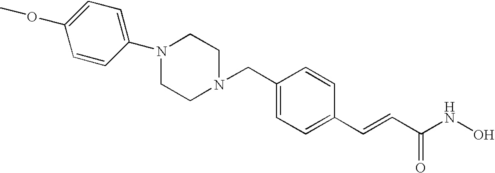 | 368 |
| 218 | 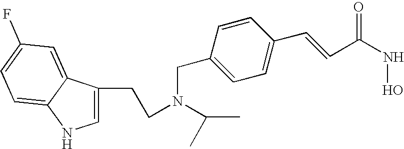 | 396 |
| 219 | 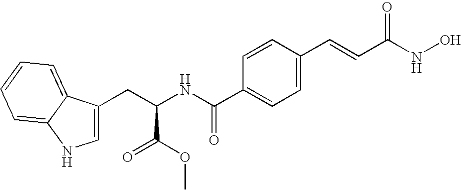 | |
| 220 | 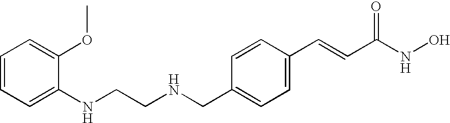 | 342 |
| 221 | 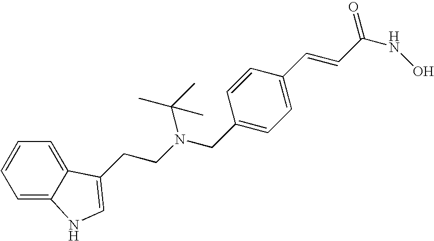 | 392 |
| 222 | 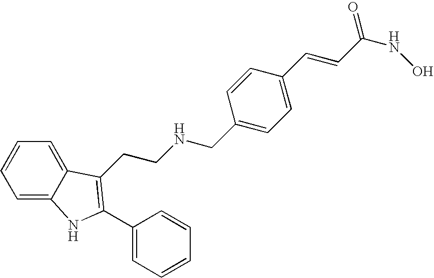 | 412 |
| 223 | 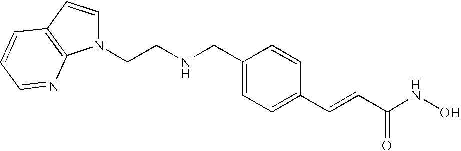 | 337 |
| 224 | 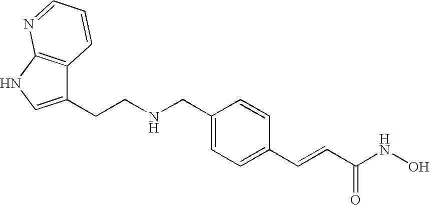 | 337 |
| 225 | 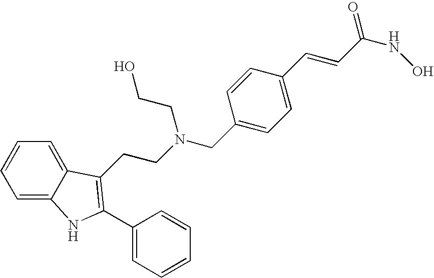 | 456 |
| 226 | 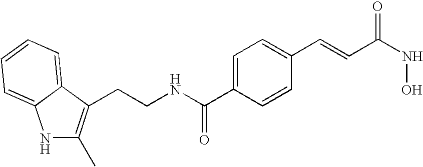 | 364 |
| 227 | 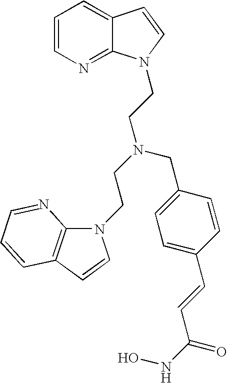 | 481 |
| 228 | 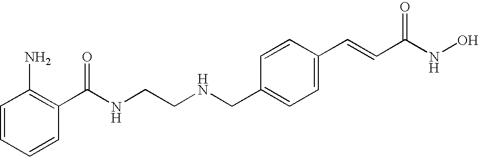 | 355 |
| 229 | 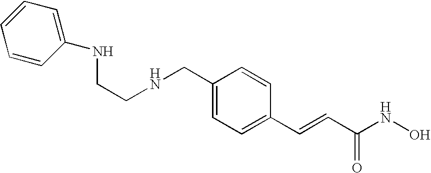 | 312 |
| 230 | 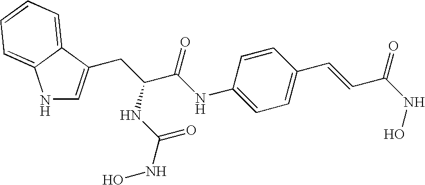 | 424 |
| 231 | 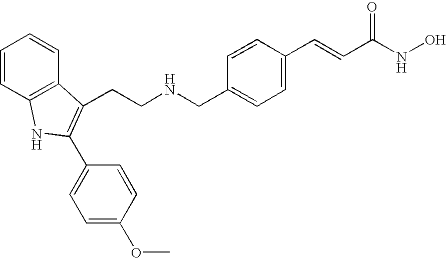 | |
| 232 | 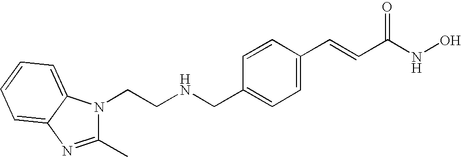 | 351 |
| 233 | 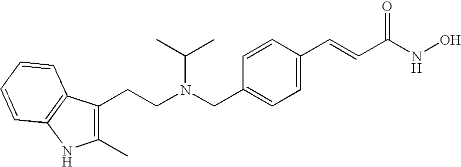 | 392 |
| 234 | 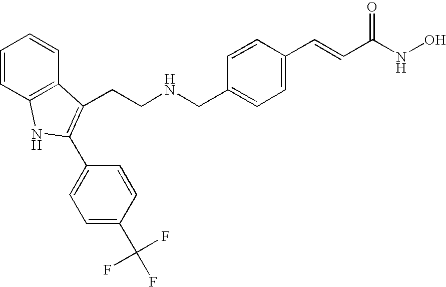 | |
| 235 | 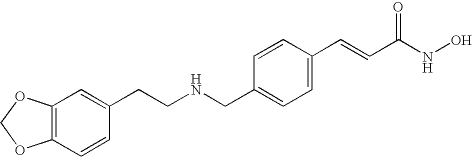 | |
| 236 | 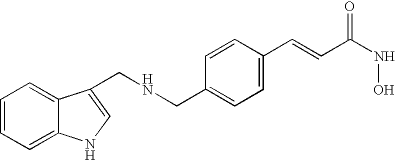 | 322 |
| 237 | 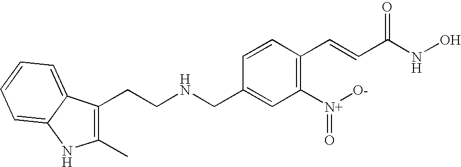 | |
| 238 | 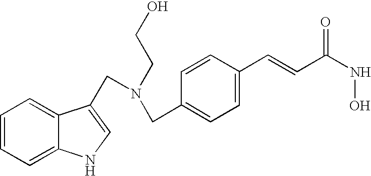 | 366 |
| 239 | 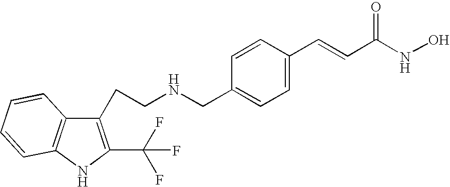 | |
| 240 | 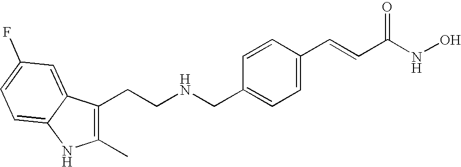 | 368 |
| 241 | 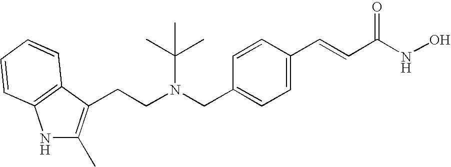 | |
| 242 | 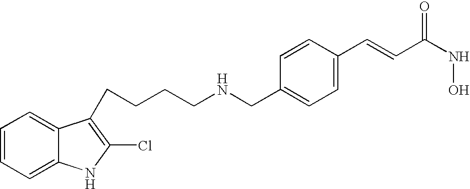 | 406 |
| 243 | 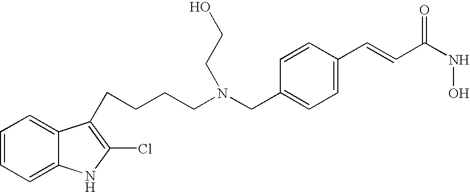 | 398 |
| 244 | 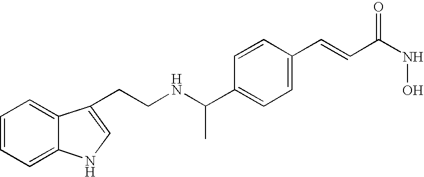 | 442 |
| 245 | 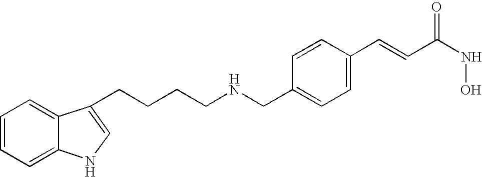 | 350 |
| 246 | 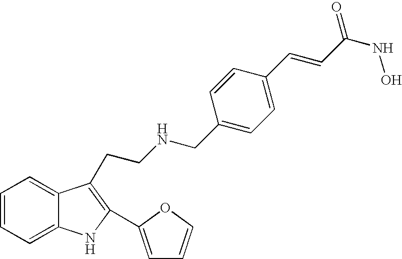 | 364 |
| 247 | 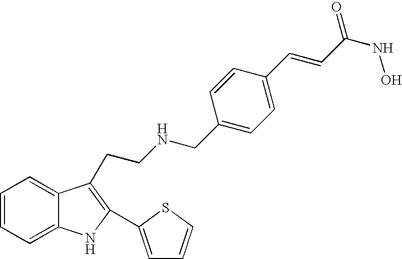 | 402 |
| 248 | 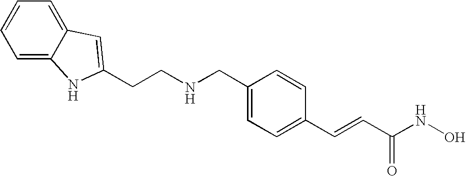 | 418 |
| 249 | 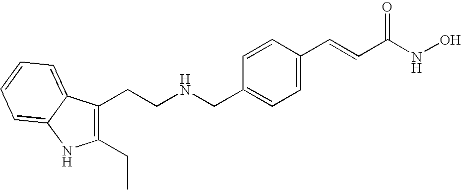 | 364 |
| 250 | 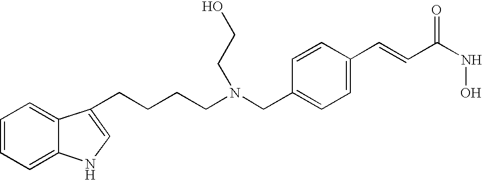 | |
| 251 | 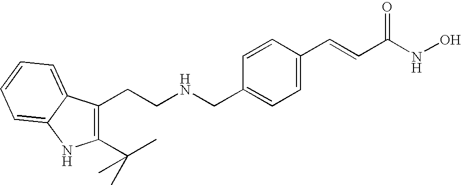 | 408 |
| 252 | 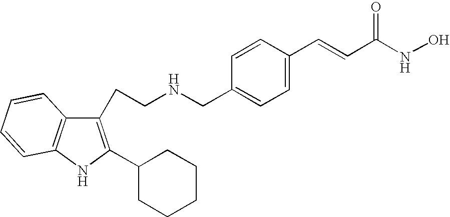 | |
| 253 | 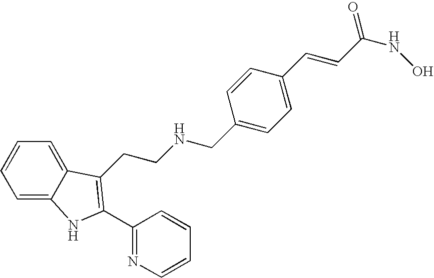 | |
| 254 | 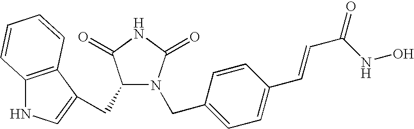 | 413 |
| 255 | 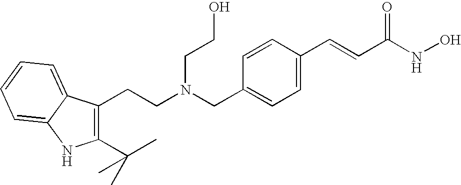 | 405 |
| 256 | 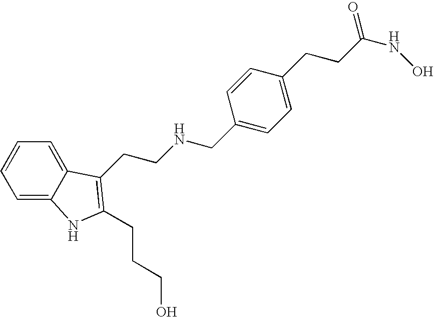 | |
| 257 | 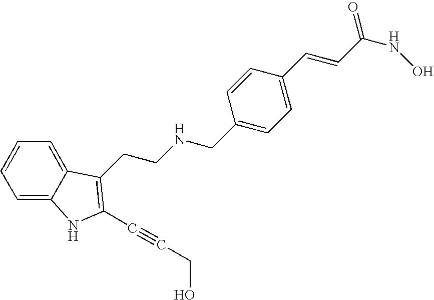 | 394 |
| 258 | 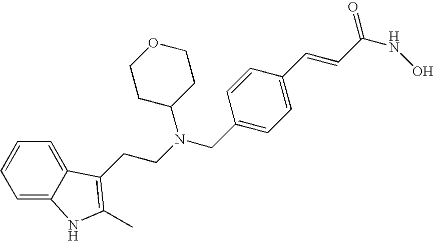 | 390 |
| 259 | 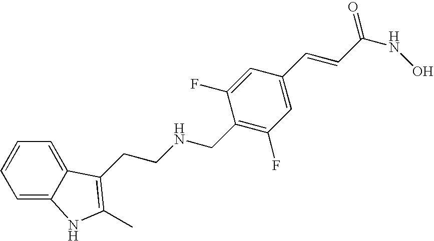 | 434 |
| 260 | 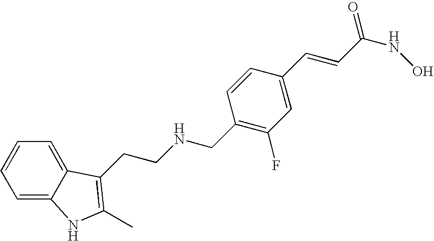 | 386 |
| 261 | 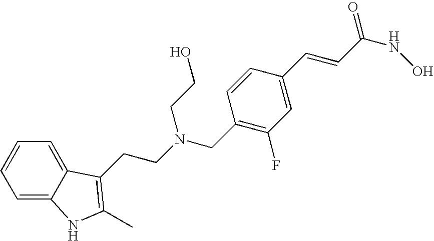 | 368 |
| 262 | 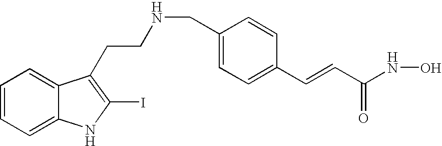 | 412 |
| 263 | 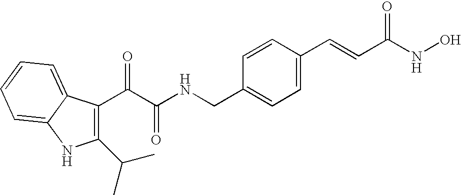 | 406 |
| 264 | 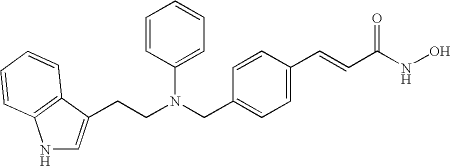 | |
| 265 | 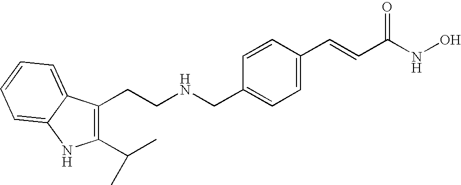 | 378 |

### EXAMPLE B1

The ascending or transverse aortic-banded mouse models are used as pressure-overload models to ascertain the beneficial effects of the inventive agents (test agents) on pathological cardiac hypertrophy. The methods described by Tarnavski et al. (2004) or Ogita et al. (2004) are used for this purpose. Briefly, anesthetized C57BL/6 male mice (age, 11 to 12 weeks) are subjected to the surgical procedure of ascending or transverse aortic banding. Sham-operated mice are subjected to similar surgical procedures without constriction of the aorta.

Blood pressure and heart rate are measured non-invasively in conscious animals before and periodically after surgery by the tail-cuff plethysmography method. Under light anesthesia, 2-dimensional guided M-mode echocardiography is performed. The percentage of left ventricular fractional shortening is calculated as [(LVDD−LVSD)/LVDD]×100(%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3−(LVDD)₃] (mg), where PWTD indicates diastolic posterior wall thickness, and VSTD indicates diastolic ventricular septal thickness.

After the above assessments, the animals are randomly segregated into aortic-banding or sham-operated groups. At the end of the aortic-banding operation, the animals are assigned to either the control (vehicle-treated) group or to the test (drug-treated) group. All groups are followed for not less than 4 weeks before using them for data analysis.

Hearts are excised after the mice are euthanized with an overdose injection of an anesthetic. Ratios of heart weight to body weight are ascertained. Sections of the hearts are prepared as previously described by Tarnavski et al. (2004), stained with hematoxylin-eosin and Masson's trichrome and observed under light microscopy.

### EXAMPLE B2

The beneficial effects of the inventive agents on cardiac hypertrophy are also ascertained in mice subjected to chronic infurion with an adrenoreceptor agonist. In these studies, male C57B1/6 mice (22-26 g) are surgically implanted with osmotic mini-pumps delivering isoproterenol (30 mg/kg/day) for periods not less than 14 days to induce cardiac hypertrophy. Control animals receive vehicle-loaded mini-pumps.

Blood pressure and heart rate are measured non-invasively in conscious animals before and periodically after surgery by the tail-cuff plethysmography method. Under light anesthesia, 2-dimensional guided M-mode echocardiography is performed. The percentage of left ventricular fractional shortening is calculated as [(LVDD −LVSD)/LVDD]×100(%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3−(LVDD)₃] (mg), where PWTD indicates diastolic posterior wall thickness, and VSTD indicates diastolic ventricular septal thickness.

After the above assessments, the animals are randomly segregated into mini-pump implanted (vehicle/drug) or sham-operated groups. All groups are followed for not less than 14 days before using them for data analysis.

Hearts are excised after the mice are euthanized with an overdose injection of an anesthetic. Ratios of heart weight to body weight are ascertained. Transverse sections of the hearts are prepared as previously described by Tarnavski et al. (2004), stained with hematoxylin-eosin and Masson's trichrome and observed under light microscopy.

### EXAMPLE B3

The beneficial effects of the inventive compounds on cardiac hypertrophy and heart failure are ascertained in a murine model of myocardial infarction and heart failure. Myocardial infarction is induced in mice (age, 11-12 weeks) by ligating the left anterior descending (LAD) coronary artery under anesthesia as described by Tarnavski et al. (2004). Sham operated animals undergo the same experimental procedures but without coronary ligation.

Blood pressure and heart rate are measured non-invasively in conscious animals before and periodically after surgery by the tail-cuff plethysmography method. Under light anesthesia, 2-dimensional guided M-mode echocardiography is performed. The percentage of left ventricular fractional shortening is calculated as [(LVDD-LVSD)/LVDD]×100(%) as described by Ogita et al. (2004). LVDD and LVSD indicate left ventricular end-diastolic and end-systolic chamber dimensions, respectively. Left ventricular mass was calculated as 1.055[(LVDD+PWTD+VSTD)3−(LVDD)₃] (mg), where PWTD indicates diastolic posterior wall thickness, and VSTD indicates diastolic ventricular septal thickness.

An invasive method for blood pressure measurement is used prior to the animal sacrifice. A micromanometer tipped Millar catheter (1.4 French) is inserted into the right carotid artery and advanced into the LV chamber to measure LV pressure.

After the above assessments, the animals (ligated, sham operated) are segregated into 2 groups and treated with the inventive compounds or corresponding vehicles. All groups are followed for not less than 14 days before using them for data analysis.

Hearts are excised after the mice are euthanized with an overdose injection of an anesthetic. Ratios of heart weight to body weight are ascertained. Transverse sections of the hearts are prepared as previously described by Tarnavski et al. (2004), stained with hematoxylin-eosin and Masson's trichrome and observed under light microscopy.

### EXAMPLE B4

The beneficial effects of the inventive compounds on cardiac hypertrophy induced by tachycardia in dogs are also ascertained. The techniques described by Motte et al. (2003) with minor modifications are used in these studies. Briefly, a bipolar pacemaker lead is surgically advanced through the right jugular vein and implanted in the right ventricular apex of anesthetized mongrel dogs. A programmable pulse generator is inserted into a subcuticular cervical pocket and connected to the pacemaker lead.

The animals undergo a pacing protocol with a stepwise increase of stimulation frequencies as described by Motte et al. (2003). Pacing is initiated by activating the pulse generator at 180 beats/min and continued for 1 week, followed by 200 beats/min over a second week, 220 beats/min over a third week, and finally 240 beats/min over the last 2 wk. The investigations are carried out at baseline (week 0) and once weekly throughout the pacing period (i.e., from week 1 to week 5). On the third day of pacing, the test agent or matching placebo is administered and continued on the same daily dose until the end of the study at five weeks.

Body weight, rectal temperature, heart rate (HR), respiratory rate (RR), and blood pressure is monitored. Doppler echocardiography is performed under continuous ECG monitoring with a 3.5- to 5-MHz mechanical sector probe. Left ventricular internal end-diastolic (LVIDd) and systolic diameters (LVIDs) as well as systolic and diastolic left ventricular free wall (LVFWs and LVFWd) and interventricular septum thickness (IVSs and IVSd) are determined. An image of the aortic flow is obtained by pulsed-wave Doppler. The velocity spectra are used to measure the preejection period (PEP) and left ventricular ejection time (LVET). From these data, left ventricular end-diastolic (EDV) and systolic volume (ESV), left ventricular ejection fraction (LVEF), and mean velocity of circumferential fiber shortening (MVCF) are calculated.

### REFERENCES

Kook H, Lepore J J, Gitler A D, Lu M M, Wing-Man Yung W, Mackay J, Zhou R, Ferrari V, Gruber P, Epstein J A. Cardiac hypertrophy and histone deacetylase-dependent transcriptional repression mediated by the atypical homeodomain protein Hop. J Clin Invest. 2003; 112:863-71.
Motte S, van Beneden R, Mottet J, Rondelet B, Mathieu M, Havaux X, Lause P, Clercx C, Ketelslegers J M, Naeije R, McEntee K. Early activation of cardiac and renal endothelin systems in experimental heart failure. Am J Physiol Heart Circ Physiol. 2003; 285(6):H2482-91.
Ogita H, Node K, Liao Y, Ishikura F, Beppu S, Asanuma H, Sanada S, Takashima S, Minamino T, Hori M, Kitakaze M. Raloxifene prevents cardiac hypertrophy and dysfunction in pressure-overloaded mice. Hypertension 2004; 43:237-42
Tarnavski O, McMullen J R, Schinke M, Nie Q, Kong S, Izumo S. Mouse cardiac surgery: comprehensive techniques for the generation of mouse models of human diseases and their application for genomic studies. Physiol Genomics. 2004; 16:349-60.

What is claimed is:

1. A method for treating and/or preventing pathologic cardiac hypertrophy and heart failure in a mammal which comprises administering to said mammal a compound of the formula (I) 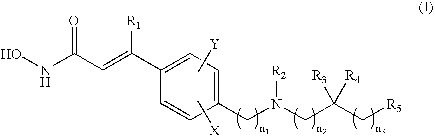
wherein
R₁ is H, halo, or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C4-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄;
m is an integer selected from 0 to 6; and
Z is selected from O, NR₁₃, S and S(O);
or a pharmaceutically acceptable salt thereof.

2. The method of claim 1 wherein the compound of formula (I) is selected from the group consisting of N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof.
